SEQUENCE LISTING (63 sequences)

SEQ ID NO: 1 - DNA, 18 bp, Artificial sequence
  Single strand DNA oligonucleotide
cgcggcataacgacccag

SEQ ID NO: 2 - DNA, 20 bp, Artificial sequence
  Single strand DNA oligonucleotide
ttccccttcgtcccagtgac

SEQ ID NO: 3 - DNA, 18 bp, Artificial sequence
  Single strand DNA oligonucleotide
ccggctgaacatcatcca

SEQ ID NO: 4 - DNA, 21 bp, Artificial sequence
  Single strand DNA oligonucleotide
agcatcggacacaggtataga

SEQ ID NO: 5 - DNA, 19 bp, Artificial sequence
  Single strand DNA oligonucleotide
tctcctctcttggcatcga

SEQ ID NO: 6 - DNA, 19 bp, Artificial sequence
  Single strand DNA oligonucleotide
cggtccccggtagcattga

SEQ ID NO: 7 - DNA, 19 bp, Artificial sequence
  Single strand DNA oligonucleotide
caaaggcatccaggccagg

SEQ ID NO: 8 - DNA, 20 bp, Artificial sequence
  Single strand DNA oligonucleotide
tgctcttcgtccaggtggtt

SEQ ID NO: 9 - DNA, 20 bp, Artificial sequence
  Single strand DNA oligonucleotide
ttcacagtgacactggacca

SEQ ID NO: 10 - DNA, 21 bp, Artificial sequence
  Single strand DNA oligonucleotide
agtatgtaagttcatggggag

SEQ ID NO: 11 - DNA, 19 bp, Artificial sequence
  Single strand DNA oligonucleotide
gagtgggcagctgagtggg

SEQ ID NO: 12 - DNA, 20 bp, Artificial sequence
  Single strand DNA oligonucleotide
gcccagcagatctcatgtcc

SEQ ID NO: 13 - DNA, 20 bp, Artificial sequence
  Single strand DNA oligonucleotide
cagatctcatgctggggagc

SEQ ID NO: 14 - DNA, 19 bp, Artificial sequence
  Single strand DNA oligonucleotide
aggaccgccatggtgagtg

SEQ ID NO: 15 - DNA, 18 bp, Artificial sequence
  Single strand DNA oligonucleotide
gctcgggcttcggaacac

SEQ ID NO: 16 - DNA, 17 bp, Artificial sequence
  Single strand DNA oligonucleotide
cccacatgcggctcgtg

SEQ ID NO: 17 - DNA, 20 bp, Artificial sequence
  Single strand DNA oligonucleotide
ccgtcttcccctccatcgtg

SEQ ID NO: 18 - DNA, 21 bp, Artificial sequence
  Single strand DNA oligonucleotide
gggcgacgtagcacagcttct

SEQ ID NO: 19 - DNA, 21 bp, Artificial sequence
  Single strand DNA oligonucleotide
tggaagaggatcaggagctgg

SEQ ID NO: 20 - DNA, 22 bp, Artificial sequence
  Single strand DNA oligonucleotide
catgagctcattcaggcctctc

SEQ ID NO: 21 - DNA, 19 bp, Artificial sequence
  Single strand DNA oligonucleotide
gtgcggctggagtgtgtgg

SEQ ID NO: 22 - DNA, 20 bp, Artificial sequence
  Single strand DNA oligonucleotide
ctccttgagcctctccacgg

SEQ ID NO: 23 - DNA, 20 bp, Artificial sequence
  Single strand DNA oligonucleotide
cctgaatcacatcgagccac

SEQ ID NO: 24 - DNA, 20 bp, Artificial sequence
  Single strand DNA oligonucleotide
tggatgcggattgtaccctt

SEQ ID NO: 25 - DNA, 20 bp, Artificial sequence
  Single strand DNA oligonucleotide
tcgctaaactgcatcgtcgc

SEQ ID NO: 26 - DNA, 20 bp, Artificial sequence
  Single strand DNA oligonucleotide
aggttgtggtcattctctgg

SEQ ID NO: 27 - DNA, 18 bp, Artificial sequence
  Single strand DNA oligonucleotide
gggcgtgttcaccagctc

SEQ ID NO: 28 - DNA, 19 bp, Artificial sequence
  Single strand DNA oligonucleotide
gccagaaggaagaccagcc

SEQ ID NO: 29 - DNA, 20 bp, Artificial sequence
  Single strand DNA oligonucleotide
gcttcgtggccgctgtggtg

SEQ ID NO: 30 - DNA, 20 bp, Artificial sequence
  Single strand DNA oligonucleotide
ctgctctgggctctggatga

SEQ ID NO: 31 - DNA, 18 bp, Artificial sequence
  Single strand DNA oligonucleotide
aggggggagccaaaaggg

SEQ ID NO: 32 - DNA, 21 bp, Artificial sequence
  Single strand DNA oligonucleotide
gaggagtgggtgtcgctgttg

SEQ ID NO: 33 - DNA, 20 bp, Artificial sequence
  Single strand DNA oligonucleotide
gatcatcttctcgaaccccg

SEQ ID NO: 34 - DNA, 18 bp, Artificial sequence
  Single strand DNA oligonucleotide
tggcaggggctcttgatg

SEQ ID NO: 35 - DNA, 21 bp, Artificial sequence
  Single strand DNA oligonucleotide
tggtcccaggaagacatgctg

SEQ ID NO: 36 - DNA, 21 bp, Artificial sequence
  Single strand DNA oligonucleotide
aggttgctcatctcagggtgg

SEQ ID NO: 37 - DNA, 21 bp, Artificial sequence
  Single strand DNA oligonucleotide
agctccccactcatcctcaag

SEQ ID NO: 38 - DNA, 19 bp, Artificial sequence
  Single strand DNA oligonucleotide
tccatctggagctgcagcc

SEQ ID NO: 39 - DNA, 30 bp, Artificial sequence
  Single strand DNA oligonucleotide
tatagctagccaccatggagtaccaggatg

SEQ ID NO: 40 - DNA, 27 bp, Artificial sequence
  Single strand DNA oligonucleotide
ttaagaattcgccttggctactgggac

SEQ ID NO: 41 - DNA, 18 bp, Artificial sequence
  Single strand DNA oligonucleotide
gagtgggctgcagggctc

SEQ ID NO: 42 - DNA, 21 bp, Artificial sequence
  Single strand DNA oligonucleotide
aggccatgagtgtcaggaagc

SEQ ID NO: 43 - DNA, 20 bp, Artificial sequence
  Single strand DNA oligonucleotide
ccaagagaaggaagcctgtg

SEQ ID NO: 44 - DNA, 21 bp, Artificial sequence
  Single strand DNA oligonucleotide
agcatcggacacaggtataga

SEQ ID NO: 45 - DNA, 21 bp, Artificial sequence
  Single strand DNA oligonucleotide
gcaccaacatcatcagcaagg

SEQ ID NO: 46 - DNA, 19 bp, Artificial sequence
  Single strand DNA oligonucleotide
cggtccccggtagcattga

SEQ ID NO: 47 - DNA, 246 bp, Homo sapiens
  misc_feature: Skipping of FPGS Exon 12, obtained sequence
cccacatgcggctcgtggccccgaggttcgagtcttgctcttcaatgctaccggggaccgggacccggcggccctgctgaagctgctgcagccctgccagtttgactatgccgtcttctgccctaacctgacagaggtgtcatccacaggcaacgcagaccaacagaacttcacagtgacactggaccaggtcctgctccgctgcctggaacaccagcagcactggaaccacctggacgaagagca

SEQ ID NO: 48 - DNA, 416 bp, Homo sapiens
  misc_feature: Insertion of FPGS Intron 10, obtained sequence
aggaccgccatggtgagtgggcagctgagtgggcaggcaggtgggtggcacctgtggagcctgcctaggagggtcccggacacacttggtctcacacaccccgcaggtgctggggagccaaaggcatccaggccagggctcctgtggcagctgcccctggcacctgtgttccagcccacatcccacatgcggctcgagcccacatcccacatgcggctcgggcttcggaacacggagtggccgggccggacgcaggtgctgcggcgcgggcccctcacctggtacctggacggtgcgcacaccgccagcagcgcgcaggcctgcgtgcgctggttccgccaggcgctgcagggccgcgagaggccgagcggtggccccgaggttcgagtcttgctcttcaatgctaccggggaccg

SEQ ID NO: 49 - DNA, 248 bp, Homo sapiens
  misc_feature: Skipping of FPGS Exon 3, obtained sequence
gagtgggctgcagggctccacctgtgccttcacggaatgtatcctccgaagctatggcctgaagacgggattctttagctctccccacctggtgcaggttcgggagcggatccgcatcaatgggcagcccatcagtcctgagctcttcaccaagtacttctggcgcctctaccaccggctggaggagaccaaggatggcagctgtgtctccatgcccccctacttccgcttcctgacactcatggcct

SEQ ID NO: 50 - DNA, 217 bp, Homo sapiens
  misc_feature: Skipping of FPGS Exon 7, obtained sequence
ccaagagaaggaagcctgtggtgtgcggagtctcctctcttggcatcgaccacaccagcctcctgggggatacggtggagaagatcgcatggcagaaagggggcatctttaagcaaggtgtccctgccttcactgtgctccaacctgaaggtcccctggcagtgctgagggaccgagcccagcagatctcatgtcctctatacctgtgtccgatgct

SEQ ID NO: 51 - DNA, 296 bp, Homo sapiens
  misc_feature: Skipping of FPGS Exon 10, obtained sequence
cagatctcatgtgctggggagccaaaggcatccaggccagggctcctgtggcagctgcccctggcacctgtgttccagcccacatcccacatgcggctcgggcttcggaacacggagtggccgggccggacgcaggtgctgcggcgcgggcccctcacctggtacctggacggtgcgcacaccgccagcagcgcgcaggcctgcgtgcgctggttccgccaggcgctgcagggccgcgagaggccgagcggtggccccgaggttcgagtcttgctcttcaatgctaccggggaccg

SEQ ID NO: 52 - DNA, 900 bp, Homo sapiens
  misc_feature: Insertion of FPGS Intron 10, Open Reading Frame
atggagtaccaggatgccgtgcgcatgctcaataccctgcagaccaatgccggctacctggagcaggtgaagcgccagcggggtgaccctcagacacagttggaagccatggaactgtacctggcacggagtgggctgcaggtggaggacttggaccggctgaacatcatccacgtcactgggacgaaggggaagggctccacctgtgccttcacggaatgtatcctccgaagctatggcctgaagacgggattctttagctctccccacctggtgcaggttcgggagcggatccgcatcaatgggcagcccatcagtcctgagctcttcaccaagtacttctggcgcctctaccaccggctggaggagaccaaggatggcagctgtgtctccatgcccccctacttccgcttcctgacactcatggccttccacgtcttcctccaagagaaggtggacctggcagtggtggaggtgggcattggcggggcttatgactgcaccaacatcatcaggaagcctgtggtgtgcggagtctcctctcttggcatcgaccacaccagcctcctgggggatacggtggagaagatcgcatggcagaaagggggcatctttaagcaaggtgtccctgccttcactgtgctccaacctgaaggtcccctggcagtgctgagggaccgagcccagcagatctcatgtcctctatacctgtgtccgatgctggaggccctcgaggaaggggggccgccgctgaccctgggcctggagggggagcaccagcggtccaacgccgccttggccttgcagctggcccactgctggctgcagcggcaggaccgccatggtgagtgggcagctgagtgggcaggcaggtgggtggcacctgtggagcctgcctag

SEQ ID NO: 53 - DNA, 1584 bp, Homo sapiens
  misc_feature: Skipping of FPGS Exon 3, Open Reading Frame
atggagtaccaggatgccgtgcgcatgctcaataccctgcagaccaatgccggctacctggagcaggtgaagcgccagcggggtgaccctcagacacagttggaagccatggaactgtacctggcacggagtgggctgcagggctccacctgtgccttcacggaatgtatcctccgaagctatggcctgaagacgggattctttagctctccccacctggtgcaggttcgggagcggatccgcatcaatgggcagcccatcagtcctgagctcttcaccaagtacttctggcgcctctaccaccggctggaggagaccaaggatggcagctgtgtctccatgcccccctacttccgcttcctgacactcatggccttccacgtcttcctccaagagaaggtggacctggcagtggtggaggtgggcattggcggggcttatgactgcaccaacatcatcaggaagcctgtggtgtgcggagtctcctctcttggcatcgaccacaccagcctcctgggggatacggtggagaagatcgcatggcagaaagggggcatctttaagcaaggtgtccctgccttcactgtgctccaacctgaaggtcccctggcagtgctgagggaccgagcccagcagatctcatgtcctctatacctgtgtccgatgctggaggccctcgaggaaggggggccgccgctgaccctgggcctggagggggagcaccagcggtccaacgccgccttggccttgcagctggcccactgctggctgcagcggcaggaccgccatggtgctggggagccaaaggcatccaggccagggctcctgtggcagctgcccctggcacctgtgttccagcccacatcccacatgcggctcgggcttcggaacacggagtggccgggccggacgcaggtgctgcggcgcgggcccctcacctggtacctggacggtgcgcacaccgccagcagcgcgcaggcctgcgtgcgctggttccgccaggcgctgcagggccgcgagaggccgagcggtggccccgaggttcgagtcttgctcttcaatgctaccggggaccgggacccggcggccctgctgaagctgctgcagccctgccagtttgactatgccgtcttctgccctaacctgacagaggtgtcatccacaggcaacgcagaccaacagaacttcacagtgacactggaccaggtcctgctccgctgcctggaacaccagcagcactggaaccacctggacgaagagcaggccagcccggacctctggagtgcccccagcccagagcccggtgggtccgcatccctgcttctggcgccccacccaccccacacctgcagtgccagctccctcgtcttcagctgcatttcacatgccttgcaatggatcagccaaggccgagaccccatcttccagccacctagtcccccaaagggcctcctcacccaccctgtggctcacagtggggccagcatactccgtgaggctgctgccatccatgtgctagtcactggcagcctgcacctggtgggtggtgtcctgaagctgctggagcccgcactgtcccagtag

SEQ ID NO: 54 - DNA, 555 bp, Homo sapiens
  misc_feature: Skipping of FPGS Exon 7, Open Reading Frame
atggagtaccaggatgccgtgcgcatgctcaataccctgcagaccaatgccggctacctggagcaggtgaagcgccagcggggtgaccctcagacacagttggaagccatggaactgtacctggcacggagtgggctgcaggtggaggacttggaccggctgaacatcatccacgtcactgggacgaaggggaagggctccacctgtgccttcacggaatgtatcctccgaagctatggcctgaagacgggattctttagctctccccacctggtgcaggttcgggagcggatccgcatcaatgggcagcccatcagtcctgagctcttcaccaagtacttctggcgcctctaccaccggctggaggagaccaaggatggcagctgtgtctccatgcccccctacttccgcttcctgacactcatggccttccacgtcttcctccaagagaaggaagcctgtggtgtgcggagtctcctctcttggcatcgaccacaccagcctcctgggggatacggtggagaagatcgcatggcagaaagggggcatctttaa

SEQ ID NO: 55 - DNA, 1002 bp, Homo sapiens
  misc_feature: Skipping of FPGS Exon 10, Open Reading Frame
atggagtaccaggatgccgtgcgcatgctcaataccctgcagaccaatgccggctacctggagcaggtgaagcgccagcggggtgaccctcagacacagttggaagccatggaactgtacctggcacggagtgggctgcaggtggaggacttggaccggctgaacatcatccacgtcactgggacgaaggggaagggctccacctgtgccttcacggaatgtatcctccgaagctatggcctgaagacgggattctttagctctccccacctggtgcaggttcgggagcggatccgcatcaatgggcagcccatcagtcctgagctcttcaccaagtacttctggcgcctctaccaccggctggaggagaccaaggatggcagctgtgtctccatgcccccctacttccgcttcctgacactcatggccttccacgtcttcctccaagagaaggtggacctggcagtggtggaggtgggcattggcggggcttatgactgcaccaacatcatcaggaagcctgtggtgtgcggagtctcctctcttggcatcgaccacaccagcctcctgggggatacggtggagaagatcgcatggcagaaagggggcatctttaagcaaggtgtccctgccttcactgtgctccaacctgaaggtcccctggcagtgctgagggaccgagcccagcagatctcagtgctggggagccaaaggcatccaggccagggctcctgtggcagctgcccctggcacctgtgttccagcccacatcccacatgcggctcgggcttcggaacacggagtggccgggccggacgcaggtgctgcggcgcgggcccctcacctggtacctggacggtgcgcacaccgccagcagcgcgcaggcctgcgtgcgctggttccgccaggcgctgcagggccgcgagaggccgagcggtggccccgaggttcgagtcttgctcttcaatgctaccggggaccgggacccggcggccctgctga

SEQ ID NO: 56 - DNA, 999 bp, Homo sapiens
  misc_feature: Skipping of FPGS Exon 12, Open Reading Frame
atggagtaccaggatgccgtgcgcatgctcaataccctgcagaccaatgccggctacctggagcaggtgaagcgccagcggggtgaccctcagacacagttggaagccatggaactgtacctggcacggagtgggctgcaggtggaggacttggaccggctgaacatcatccacgtcactgggacgaaggggaagggctccacctgtgccttcacggaatgtatcctccgaagctatggcctgaagacgggattctttagctctccccacctggtgcaggttcgggagcggatccgcatcaatgggcagcccatcagtcctgagctcttcaccaagtacttctggcgcctctaccaccggctggaggagaccaaggatggcagctgtgtctccatgcccccctacttccgcttcctgacactcatggccttccacgtcttcctccaagagaaggtggacctggcagtggtggaggtgggcattggcggggcttatgactgcaccaacatcatcaggaagcctgtggtgtgcggagtctcctctcttggcatcgaccacaccagcctcctgggggatacggtggagaagatcgcatggcagaaagggggcatctttaagcaaggtgtccctgccttcactgtgctccaacctgaaggtcccctggcagtgctgagggaccgagcccagcagatctcatgtcctctatacctgtgtccgatgctggaggccctcgaggaaggggggccgccgctgaccctgggcctggagggggagcaccagcggtccaacgccgccttggccttgcagctggcccactgctggctgcagcggcaggaccgccatggtgctggggagccaaaggcatccaggccagggctcctgtggcagctgcccctggcacctgtgttccagcccacatcccacatgcggctcgtggccccgaggttcgagtcttgctcttcaatgctaccggggaccgggacccggcggccctgctga

SEQ ID NO: 57 - protein, 299 aa, Homo sapiens
  misc_feature: Insertion of FPGS Intron 10, deduced amino acid sequence
MEYQDAVRMLNTLQTNAGYLEQVKRQRGDPQTQLEAMELYLARSGLQVEDLDRLNIIHVTGTKGKGSTCAFTECILRSYGLKTGFFSSPHLVQVRERIRINGQPISPELFTKYFWRLYHRLEETKDGSCVSMPPYFRFLTLMAFHVFLQEKVDLAVVEVGIGGAYDCTNIIRKPVVCGVSSLGIDHTSLLGDTVEKIAWQKGGIFKQGVPAFTVLQPEGPLAVLRDRAQQISCPLYLCPMLEALEEGGPPLTLGLEGEHQRSNAALALQLAHCWLQRQDRHGEWAAEWAGRWVAPVEPA

SEQ ID NO: 58 - protein, 527 aa, Homo sapiens
  misc_feature: Skipping of FPGS Exon 3, deduced amino acid sequence
MEYQDAVRMLNTLQTNAGYLEQVKRQRGDPQTQLEAMELYLARSGLQGSTCAFTECILRSYGLKTGFFSSPHLVQVRERIRINGQPISPELFTKYFWRLYHRLEETKDGSCVSMPPYFRFLTLMAFHVFLQEKVDLAVVEVGIGGAYDCTNIIRKPVVCGVSSLGIDHTSLLGDTVEKIAWQKGGIFKQGVPAFTVLQPEGPLAVLRDRAQQISCPLYLCPMLEALEEGGPPLTLGLEGEHQRSNAALALQLAHCWLQRQDRHGAGEPKASRPGLLWQLPLAPVFQPTSHMRLGLRNTEWPGRTQVLRRGPLTWYLDGAHTASSAQACVRWFRQALQGRERPSGGPEVRVLLFNATGDRDPAALLKLLQPCQFDYAVFCPNLTEVSSTGNADQQNFTVTLDQVLLRCLEHQQHWNHLDEEQASPDLWSAPSPEPGGSASLLLAPHPPHTCSASSLVFSCISHALQWISQGRDPIFQPPSPPKGLLTHPVAHSGASILREAAAIHVLVTGSLHLVGGVLKLLEPALSQ

SEQ ID NO: 59 - protein, 184 aa, Homo sapiens
  misc_feature: Skipping of FPGS Exon 7, deduced amino acid sequence
MEYQDAVRMLNTLQTNAGYLEQVKRQRGDPQTQLEAMELYLARSGLQVEDLDRLNIIHVTGTKGKGSTCAFTECILRSYGLKTGFFSSPHLVQVRERIRINGQPISPELFTKYFWRLYHRLEETKDGSCVSMPPYFRFLTLMAFHVFLQEKEACGVRSLLSWHRPHQPPGGYGGEDRMAERGHL

SEQ ID NO: 60 - protein, 333 aa, Homo sapiens [sequence omitted]

SEQ ID NO: 61 - protein, 332 aa, Homo sapiens [sequence omitted]

SEQ ID NO: 62 - DNA, 1638 bp, Homo sapiens
  misc_feature: WT FPGS
atggagtaccaggatgccgtgcgcatgctcaataccctgcagaccaatgccggctacctggagcaggtgaagcgccagcggggtgaccctcagacacagttggaagccatggaactgtacctggcacggagtgggctgcaggtggaggacttggaccggctgaacatcatccacgtcactgggacgaaggggaagggctccacctgtgccttcacggaatgtatcctccgaagctatggcctgaagacgggattctttagctctccccacctggtgcaggttcgggagcggatccgcatcaatgggcagcccatcagtcctgagctcttcaccaagtacttctggcgcctctaccaccggctggaggagaccaaggatggcagctgtgtctccatgcccccctacttccgcttcctgacactcatggccttccacgtcttcctccaagagaaggtggacctggcagtggtggaggtgggcattggcggggcttatgactgcaccaacatcatcaggaagcctgtggtgtgcggagtctcctctcttggcatcgaccacaccagcctcctgggggatacggtggagaagatcgcatggcagaaagggggcatctttaagcaaggtgtccctgccttcactgtgctccaacctgaaggtcccctggcagtgctgagggaccgagcccagcagatctcatgtcctctatacctgtgtccgatgctggaggccctcgaggaaggggggccgccgctgaccctgggcctggagggggagcaccagcggtccaacgccgccttggccttgcagctggcccactgctggctgcagcggcaggaccgccatggtgctggggagccaaaggcatccaggccagggctcctgtggcagctgcccctggcacctgtgttccagcccacatcccacatgcggctcgggcttcggaacacggagtggccgggccggacgcaggtgctgcggcgcgggcccctcacctggtacctggacggtgcgcacaccgccagcagcgcgcaggcctgcgtgcgctggttccgccaggcgctgcagggccgcgagaggccgagcggtggccccgaggttcgagtcttgctcttcaatgctaccggggaccgggacccggcggccctgctgaagctgctgcagccctgccagtttgactatgccgtcttctgccctaacctgacagaggtgtcatccacaggcaacgcagaccaacagaacttcacagtgacactggaccaggtcctgctccgctgcctggaacaccagcagcactggaaccacctggacgaagagcaggccagcccggacctctggagtgcccccagcccagagcccggtgggtccgcatccctgcttctggcgccccacccaccccacacctgcagtgccagctccctcgtcttcagctgcatttcacatgccttgcaatggatcagccaaggccgagaccccatcttccagccacctagtcccccaaagggcctcctcacccaccctgtggctcacagtggggccagcatactccgtgaggctgctgccatccatgtgctagtcactggcagcctgcacctggtgggtggtgtcctgaagctgctggagcccgcactgtcccagtag

SEQ ID NO: 63 - protein, 545 aa, Homo sapiens
  misc_feature: WT FPGS
MEYQDAVRMLNTLQTNAGYLEQVKRQRGDPQTQLEAMELYLARSGLQVEDLDRLNIIHVTGTKGKGSTCAFTECILRSYGLKTGFFSSPHLVQVRERIRINGQPISPELFTKYFWRLYHRLEETKDGSCVSMPPYFRFLTLMAFHVFLQEKVDLAVVEVGIGGAYDCTNIIRKPVVCGVSSLGIDHTSLLGDTVEKIAWQKGGIFKQGVPAFTVLQPEGPLAVLRDRAQQISCPLYLCPMLEALEEGGPPLTLGLEGEHQRSNAALALQLAHCWLQRQDRHGAGEPKASRPGLLWQLPLAPVFQPTSHMRLGLRNTEWPGRTQVLRRGPLTWYLDGAHTASSAQACVRWFRQALQGRERPSGGPEVRVLLFNATGDRDPAALLKLLQPCQFDYAVFCPNLTEVSSTGNADQQNFTVTLDQVLLRCLEHQQHWNHLDEEQASPDLWSAPSPEPGGSASLLLAPHPPHTCSASSLVFSCISHALQWISQGRDPIFQPPSPPKGLLTHPVAHSGASILREAAAIHVLVTGSLHLVGGVLKLLEPALSQ

(19) United States
(12) Patent Application Publication
(10) Pub. No.: US 2011/0229892 A1
(43) Pub. Date: Sep. 22, 2011

(54) METHOD FOR PREDICTING A PATIENT'S RESPONSIVENESS TO ANTI-FOLATE THERAPY

(76) Inventors: Yehuda G Assaraf, Moshav Rakefet - Doar-Na Misgav (IL); Michal Stark, Moshav Rakefet - Doar-Na Misgav (IL)

(21) Appl. No.: 13/129,604

(22) PCT Filed: Nov. 17, 2009
(86) PCT No.: PCT/IL2009/001087

§ 371 Date: May 17, 2011

Related U.S. Application Data
(60) Provisional application No. 61/115,346, filed on Nov. 17, 2008.

(51) Int. Cl. C12Q 1/68; C12Q 1/25; G01N 33/573; C07K 16/40; C07H 21/04

(52) U.S. Cl. 435/6.11; 435/4; 435/7.1; 530/389.8; 536/23.1

(57) ABSTRACT

A method of predicting responsiveness of a subject to a folylpolyglutamate synthetase (FPGS) dependent anti-folate is provided. The method comprises analyzing for a presence or absence of a splice variant of FPGS or a polypeptide encoded thereby, in a sample of the subject, wherein the presence of the splice variant or the polypeptide encoded thereby is indicative of a negative response to a FPGS-dependent anti-folate. Kits for prediction responsiveness of a subject to FPGS-dependent anti-folate are also disclosed. Antibodies specific for splice variants, the splice variant nucleic acids and polypeptides encoded by the splice variants are also claimed. In particular splice variants missing exons selected from the group of exon 3, exon 7, exon 10 and exon 12 are disclosed.

### FIELD AND BACKGROUND OF THE INVENTION

The present invention, in some embodiments thereof, relates to methods and kits for predicting a patient's responsiveness to anti-folate therapy and kits for same. In spite of numerous advances in medical research, cancer remains a major cause of death worldwide, and there is a need for rapid and simple methods for the diagnosis of cancer in order to facilitate appropriate remedial action, as well as the identification of novel targets for cancer therapy. The availability of good diagnostic methods for cancer is also important to assess patient responses to treatment, or to assess recurrence due to re-growth at the original site or metastases.

Folates are essential vitamins that serve as one-carbon donors in a variety of biosynthetic pathways including the de novo biosynthesis of purines and thymidylate, mitochondrial protein synthesis and amino acid conversion. Folate analogues (i.e. antifolates) exert their cytotoxic activity via potent inhibition of various folate-dependent enzymes that mediate de novo nucleotide biosynthesis, block DNA replication and hence induce cell death. Consequently, antifolates are currently used as important anti-cancer drugs for the treatment of various human malignancies; in this respect, methotrexate (MTX), the first antifolate introduced nearly 60 years ago, achieved remarkable remissions in acute lymphoblastic leukemia (ALL). MTX and novel antifolates including pemetrexed (ALIMTA™) and raltitrexed (TOMUDEX™) are currently used for the treatment of various human cancers including ALL, lymphomas, breast cancer, osteosarcoma, colorectal cancer, malignant pleural mesothelioma and choriocarcinoma. Antifolates are also used, particularly upon a low dose regimen, for the treatment of non-neoplastic disorders including rheumatoid arthritis, psoriasis and Crohn's disease. Currently, antifolates are also used for the treatment of parasitic diseases and fungal diseases such as malaria and Pneumocystis Carinii pneumonia, respectively.

Folate cofactors and antifolates are divalent anions and hence require specific membrane transporters and receptors for their translocation across biological membranes. These transport routes include the proton-coupled folate transporter (PCFT/SLC46A1), the reduced folate carrier (RFC/SLC19A1) and folate receptors. Once taken up into cells, folates and antifolates undergo polyglutamylation catalyzed by folylpolyglutamate synthetase (FPGS) which adds up to 10 glutamate residues, one at a time, to the γ-carboxyl residue of folates and antifolates. This unique metabolism renders folates and antifolates polyanions which can no longer be effluxed out of cells, thereby resulting in enhanced intracellular retention. Since polyglutamylation plays a key role in cellular retention of antifolates and thus increases their cytotoxic activity, loss of FPGS activity is an established mechanism of resistance to polyglutamylation-dependent antifolates in vitro and in vivo. This phenomenon was observed in various malignant cell lines selected for resistance to intermittent exposure to high dose polyglutamatable antifolates including MTX, raltitrexed and pemetrexed. Using this modality of pulse drug exposure, the vast majority of antifolate-resistant tumor cell lines displayed a 90-99% loss of FPGS activity, frequently in the absence of any substantial decrease in FPGS mRNA levels. Although inactivating FPGS mutations may result in the loss of FPGS activity, they are relatively infrequent.

U.S. Patent Application 20040101834 teaches a method for predicting the responsiveness of a cancer patient to antifolate-containing chemotherapy by analyzing genes associated with folate metabolism or uptake for mutations.

U.S. Patent Application 20050112627 teaches a method for predicting the responsiveness of a cancer patient to antifolate-containing chemotherapy by genotyping the patient at a polymorphic site in a folate pathway gene.

### SUMMARY OF THE INVENTION

According to an aspect of some embodiments of the present invention there is provided a method of predicting responsiveness of a subject to a folylpolyglutamate synthetase (FPGS) dependent anti-folate, the method comprising analyzing for a presence or absence of a splice variant of FPGS or a polypeptide encoded thereby, in a sample of the subject, wherein the presence of the splice variant or the polypeptide encoded thereby is indicative of a negative response to a FPGS-dependent anti-folate. According to an aspect of some embodiments of the present invention there is provided a method of selecting an anti-folate for the treatment of a disease in a subject in need thereof, the method comprising analyzing for a presence or absence of a splice variant of FPGS or a polypeptide encoded thereby, in a sample of the subject, wherein the presence of the splice variant or polypeptide encoded thereby is indicative of treatment with a FPGS-independent anti-folate and the absence of the splice variant or the polypeptide encoded thereby is indicative of treatment with a FPGS-dependent anti-folate.

According to some embodiments of the invention, the sample comprises bone marrow or peripheral blood.

According to some embodiments of the invention, the sample comprises an RNA sample.

According to some embodiments of the invention, the sample comprises a protein sample.

According to some embodiments of the invention, the FPGS dependent anti-folate is selected from the group consisting of methotrexate, aminopeterin, lometrexol, edatrexate, pemetrexed and raltitrexed.

According to some embodiments of the invention, the analyzing is effected by calculating a size of an RNA encoding FPGS.

According to some embodiments of the invention, the analyzing is effected by determining a sequence of at least a portion of an RNA encoding FPGS.

According to some embodiments of the invention, the analyzing is effected using an antibody which binds to the polypeptide encoded by the splice variant of the FPGS and does not bind to a wild-type FPGS.

According to some embodiments of the invention, the sequence of at least the portion of the RNA encoding FPGS comprises at least a part of an intron selected from the group consisting of intron 1, 2, 10, 11 and 12.

According to some embodiments of the invention, the sequence of at least the portion of the RNA encoding FPGS is devoid of at least a part of an exon selected from the group consisting of exon 3, exon 7, exon 10 and exon 12.

According to some embodiments of the invention, the sequence of at least the portion of the RNA encoding FPGS is selected from the group consisting of SEQ ID NOs: 47-51.

According to some embodiments of the invention, the sequence of at least the portion of the RNA hybridizes with an RNA molecule which comprises a nucleic acid sequence as set forth in SEQ ID NOs: 16, 41 and 43.

According to some embodiments of the invention, the sequence of at least the portion of the RNA comprises a mutation in at least one of the positions selected from the group consisting of:

(i) a region bridging exon 2 and exon 3 of the FPGS RNA;

(ii) a region bridging exon 3 and exon 4 of the FPGS RNA;

(iii) a region bridging exon 6 and exon 7 of the FPGS RNA;

(iv) a region bridging exon 7 and exon 8 of the FPGS RNA;

(v) a region bridging exon 9 and exon 10 of the FPGS RNA;

(vi) a region bridging exon 10 and exon 11 of the FPGS RNA;

(vii) a region bridging exon 11 and exon 12 of the FPGS RNA; and

(viii) a region bridging exon 12 and exon 13 of the FPGS RNA.

According to some embodiments of the invention, the subject has been diagnosed with a disease selected from the group consisting of cancer, an inflammatory disease, and an autoimmune disease.

According to some embodiments of the invention, the cancer is selected from the group consisting of acute lymphoblastic leukemia (ALL), lymphomas, breast cancer, osteosarcoma, colorectal cancer, malignant pleural mesothelioma and choriocarcinoma.

According to some embodiments of the invention, the FPGS independent anti-folate is selected from the group consisting of plevitrexed, piritexim and neutrexin.

According to an aspect of some embodiments of the present invention there is provided an antibody which binds to an expression product of a splice variant of FPGS and does not bind to wild-type FPGS.

According to an aspect of some embodiments of the present invention there is provided a kit for assessing a responsiveness of a patient to antifolate therapy, the kit comprising at least one polynucleotide agent which detects a splice variant of FPGS RNA.

According to some embodiments of the invention, the at least one polynucleotide agent is encoded by a nucleic acid sequence selected from the group consisting of SEQ ID NOs: 16, 41 and 43.

According to some embodiments of the invention, the kit further comprises at least one additional agent selected from the group consisting of a reverse transcriptase enzyme, a DNA polymerase enzyme, dNTPs.

According to an aspect of some embodiments of the present invention there is provided a kit comprising the antibody of the present invention.

According to an aspect of some embodiments of the present invention there is provided an isolated polynucleotide encoding an FPGS polypeptide comprising a nucleic acid sequence at least 80% identical to at least one of the sequences selected from the group consisting of SEQ ID NO: 47-51.

According to an aspect of some embodiments of the present invention there is provided an isolated FPGS polypeptide comprising an amino acid sequence being encoded by a nucleic acid sequence at least 80% identical to at least one of the sequences selected from the group consisting of SEQ ID NO: 47-51.

According to some embodiments of the invention, the isolated FPGS polypeptide comprises an amino acid sequence as set forth in SEQ ID NOs: 57-61.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-C are photographs and diagrams revealing the presence of introns in various cDNAs as detected by RT-PCR analysis. A) Left panel, PCR was performed on the FPGS gene using two sets of forward and reverse primers; a. Primers residing within the first and third exons (EX1-up SEQ ID NO: 1 and EX3-dw SEQ ID NO: 2). RT-PCR on cDNA from CCRF-CEM cells produced the expected product size (e.g. 263 bp, lane 1), whereas the MTX^{R5}P cells cDNA yielded a markedly longer product of 1570 bp (lane 2). b. PCR was performed in order to confirm the presence of intron 10 in cDNA from MTX^{R5}P cells using a forward primer (INT10 SEQ ID NO: 11) residing within this intron and EX13-dw SEQ ID NO: 6. The white vertical line has been inserted to indicate repositioned gel lanes. Right panel, scheme illustrating the positions of the primers as well as the predicted and observed PCR products. B, C) PCR was performed on various genes using a panel of forward and reverse primers (Table 3) residing within neighbor exons in order to obtain different product sizes revealing normal and unspliced cDNAs: B) RT-PCR on cDNA from parental and MTX^{R5}P cells produced the expected product sizes. C) RT-PCR on WT cDNA produced normal size products, whereas MTX^{R5}P cell's cDNA revealed longer fragments as predicted for unspliced cDNA containing introns; all sizes of PCR fragments are depicted in Table 3.

FIG. 2 is a sample of the tracing of DNA sequencing illustrating the existence of two separate sequences obtained with two primers. PCR product was produced using primers EX8-up SEQ ID NO: 5 and Ex13-dw SEQ ID NO: 6 and then sequenced. Two nucleotides appear at the same position starting at nucleotide 362, one sequence matching the expected exon 10, whereas the other matches exon 11 ahead of its time.

FIGS. 3A-C are photographs of RT-PCR analysis revealing exon skipping and intron inclusion in the FPGS cDNA. PCR was performed on the FPGS gene using 3 sets of forward primers with EX13-dw SEQ ID NO: 6; A) Primer ex9-10 (SEQ ID NO: 12) residing in the junction between exons 9 and 10 (lanes 1, 3, 5, 7), primer ex9-11 (SEQ ID NO: 13) residing in the 3′-end of exon 9 and 5′-end of exon 11 and thus being diagnostic of exon 10 skipping (lanes 2, 4, 6, 8). B) PCR was performed in order to corroborate the presence of intron 10 in CEM-7OH MTX and Molt-4-7OH cDNA using a forward primer (ex10int10 SEQ ID NO: 14) on the junction between exon 10 and intron 10. C) TNFα as a control gene for normal splicing.

FIGS. 4A-D are photographs illustrating Northern blot analysis of FPGS mRNA levels. RNA was blotted onto Zeta-Probe nylon membrane and hybridized with a [³²P]labeled FPGS probe as detailed in Materials and Methods. A) RNA from parental CCRF-CEM and Molt-4 cells (lanes 1 and 4, respectively) as well as antifolate-resistant CEM-7OH MTX, CEM MTX^{R10} and Molt-4-7OH MTX cells (lanes 2, 3 and 5, respectively). A discrete band with an expected size of 2400 bp can be observed for both parental cell lines (lanes 1 and 4), whereas the antifolate-resistant cells CEM-7OH MTX and MTX^{R10} exhibit a smear spanning several hundreds of base pairs (lanes 2 and 3, respectively). C) RNA from parental CCRF-CEM cells and their pIRES/FPGSΔexon10 transfectant (lanes 1 and 2, respectively) as well as the antifolate-resistant cell line CEM MTX^{R10} and its pcDNA3/hFPGS transfectant (lanes 3 and 4, respectively). pIRES/FPGSΔexon10 is transcribed to yield a ˜3550 bp long operon including the FPGS gene, the IRES translation initiation sequence and the EGFP gene. B, D) The intensity of the FPGS transcript was normalized to the methylene blue staining of the 18S ribosomal RNA band.

FIG. 5 is a photograph of RT-PCR analysis revealing skipping of FPGS exon 12 in RNA from ALL patient specimens. PCR was performed on cDNA prepared from 5 ALL patients at first diagnosis (lanes 2, 4, 6, 8, 10, 12, 13, 14) and at relapse (lanes 3, 5, 7, 9, 11), using primer ex9-10 SEQ ID NO: 12 residing in the junction between exons 9 and 10 and primer EX13-dw SEQ ID NO: 6 (A), or ex11-13 SEQ ID NO: 16 residing in the junction between exons 11 and 13 and primer EX15-dw SEQ ID NO: 8 (B). A) All samples (including CCRF CEM, lane 1) exhibited the expected 450 bp long product, whereas relapse sample R1 (Lane 3) displayed low levels of another 300 by product. DNA sequencing corroborated the absence of exon 12 in this 300 bp product. B) This diagnostic PCR results in a 250 bp product only when exon 12 is absent, and was obtained for relapse sample R1 (lane 3) and to a lower extent in two diagnosis samples D5 and D8 (lanes 10 and 14, respectively).

FIGS. 6A-B are photographs of RT-PCR analysis of FPGS exon skipping in ALL patient specimens at diagnosis. RNA samples from 24 adult ALL patients were reverse-transcribed and analyzed for (A) skipping of FPGS exons 7 and 12. The diagnostic primers (detailed in Materials and Methods) amplify a 250 bp product lacking either exon 7 or 12. (B) Primers residing within FPGS exons 1 and 3 or 12 and 15 as well as GAPDH exons 6 and 8 were used for the control reactions. One patient was not analyzed for exon 7 skipping and two were not analyzed for GAPDH due to insufficient amount of RNA (lanes 1 and 19 upper and bottom panels).

FIGS. 7A-C are graphs illustrating Kaplan-Meier product limit analysis for patients' outcome. Progression free survival (A), Probability not to die of disease (B) and Overall survival (C) were analyzed for the 24 adult ALL patients in association with the various prognostic factors (i.e., age >35 years, WBC count, karyotype and FPGS exon skipping). Each section (A-C) illustrates the two prognostic factors exhibiting the most significant association with patients' outcome. The maximum follow-up time for all the plots is 62 months. Since the plots do not change in the last 12 months only 50 months of follow-up is presented.

### DESCRIPTION OF SPECIFIC EMBODIMENTS OF THE INVENTION

The present invention, in some embodiments thereof, relates to methods and kits for predicting a patient's responsiveness to anti-folate therapy.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Folate analogues (i.e. anti-folates) exert their cytotoxic activity via potent inhibition of various folate-dependent enzymes that mediate de novo nucleotide biosynthesis, block DNA replication and hence induce cell death. Consequently, anti-folates are currently used as important drugs for the treatment of various human malignancies as well as for rheumatoid arthritis, psoriasis and Crohn's disease.

Folylpoly-γ-glutamate synthetase (FPGS) catalyzes the polyglutamylation and thus intracellular retention of folates and anti-folates through the addition of multiple glutamate equivalents to their γ-carboxyl residue. Since polyglutamylation of anti-folates is crucial for their pharmacological activity, loss of FPGS function results in decreased cellular levels of polyglutamylation-dependent anti-folates and consequent drug resistance.

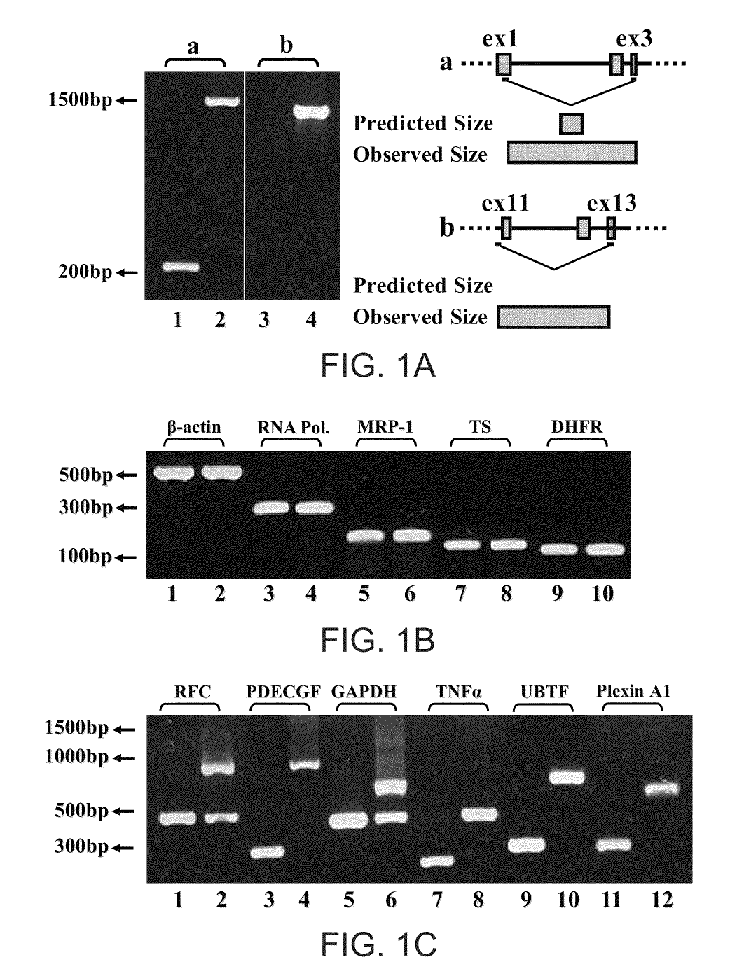

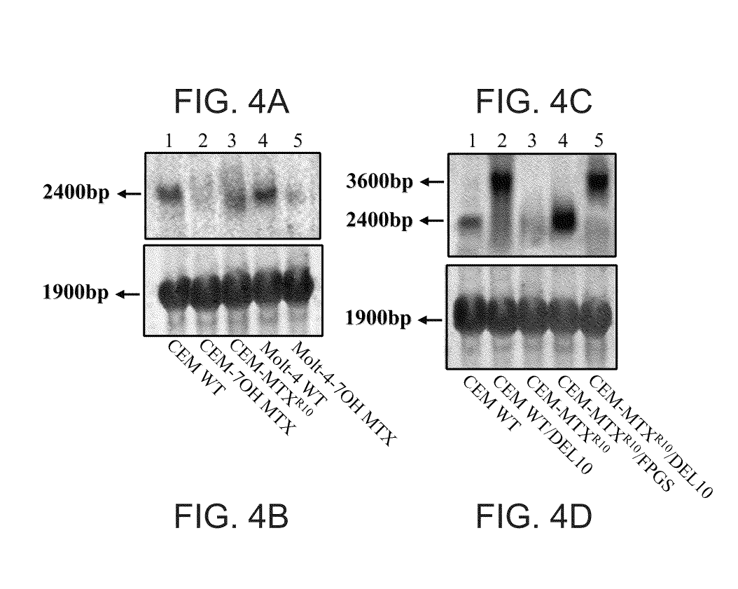

Whilst exploring the molecular basis of anti-folate-resistance, the present inventors demonstrated by RT-PCR analysis (FIGS. 1A-C and 3A-B), and Northern blot analysis (FIGS. 4A-D) that methotrexate-resistant leukemia cell lines exhibited impaired splicing of FPGS mRNA based on intron retention and/or exon skipping, thereby resulting in loss of FPGS function.

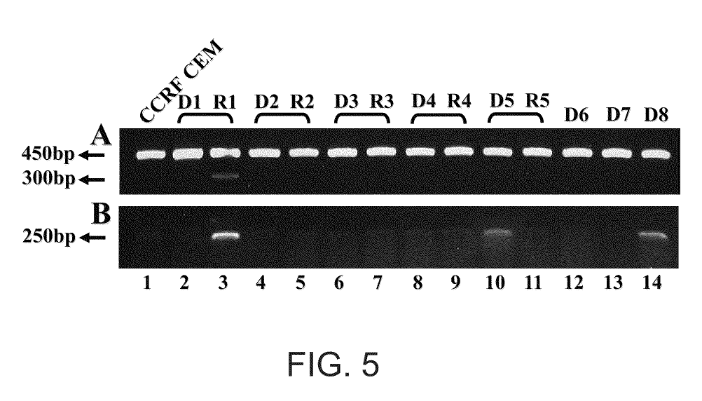

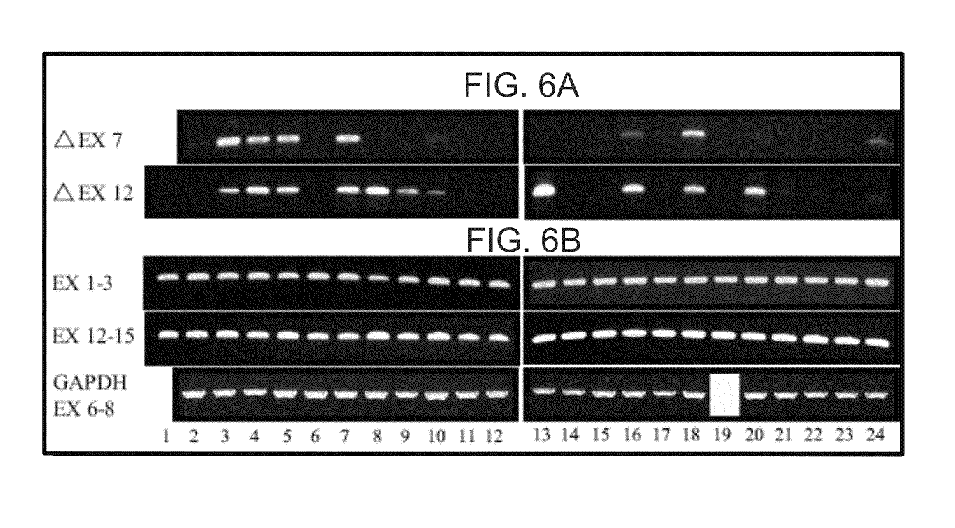

The present inventors subsequently revealed exon 7 skipping, exon 8 skipping and/or exon 12 skipping in FPGS transcripts in blood or bone marrow samples from patients with acute lymphoblastic leukemia at diagnosis (FIGS. 5 and 6A-B). In addition, the present inventors revealed exon 12 skipping at relapse, occurring after high dose MTX-containing chemotherapy (FIG. 5). These results constitute the first demonstration of the loss of FPGS function via aberrant mRNA splicing, thereby resulting in loss of anti-folate retention and drug resistance.

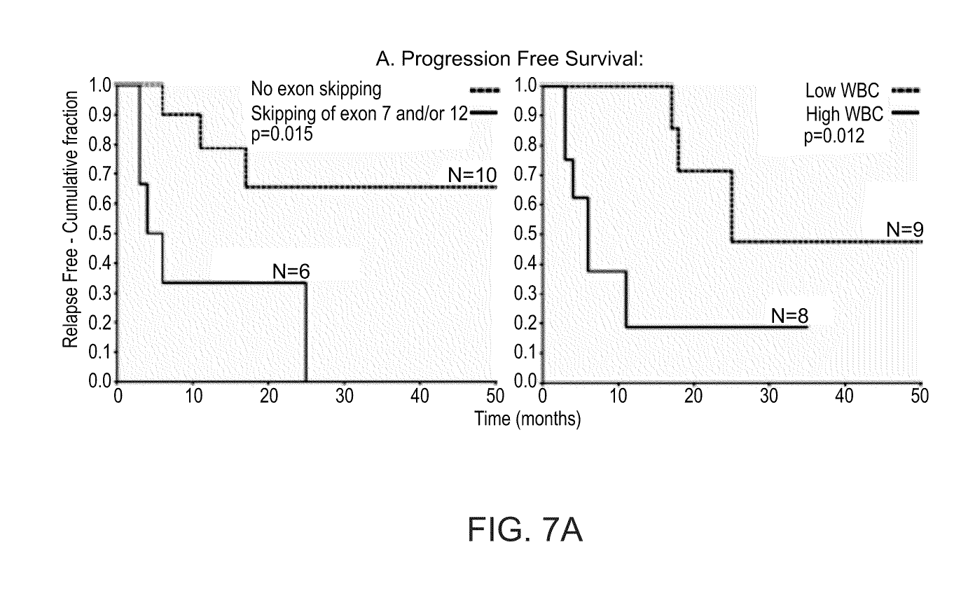

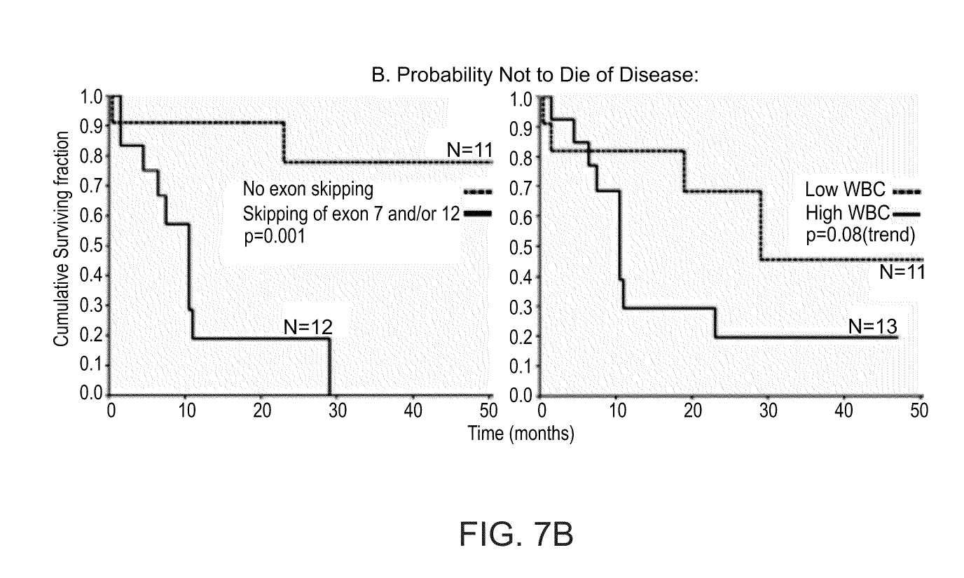

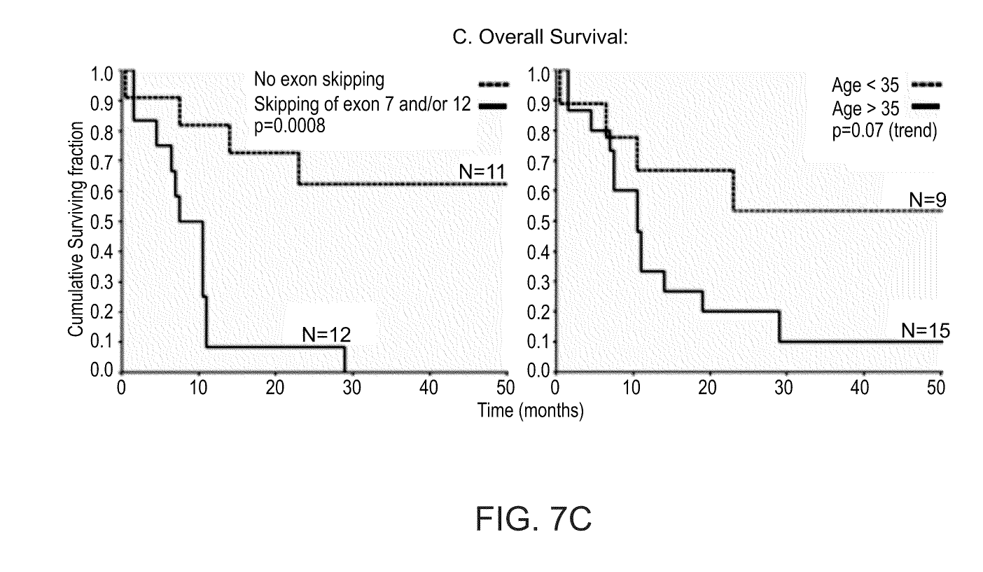

Furthermore, using statistical analysis, the present inventors demonstrated that impaired FPGS splicing appears as a strong independent factor correlated with inferior prognosis (FIGS. 7A-C).

Accordingly, the present inventors propose that detection of splice variants of FPGS may be used to predict a patient's responsiveness to anti-folate therapy and for selection of an appropriate drug treatment.

Thus, according to one aspect of the present invention, there is provided a method of predicting responsiveness of a subject to a folylpolyglutamate synthetase (FPGS) dependent anti-folate, the method comprising analyzing for a presence or absence of a splice variant of FPGS or a polypeptide encoded thereby, in a sample of the subject, wherein the presence of the splice variant or the polypeptide encoded thereby is indicative of a negative response to a FPGS-dependent anti-folate.

As used herein, the term “responsiveness” refers to either an initial response of the patient to anti-folate treatment, i.e., the response thereof to the first treatment, prior to which no exposure to anti-folate compounds has been experienced; or a secondary response of the patient to anti-folate-containing treatment, i.e., the response to subsequent treatments, prior to which exposure to anti-folate compounds has already been experienced.

According to an embodiment of this aspect of the present invention, the subject is a mammal (e.g. a human). The subject has typically been diagnosed with a disease, for which anti-folate treatment is recommended. Such diseases include, but are not limited to cancer, inflammatory diseases and autoimmune diseases.

The phrase “autoimmune disease”, as used herein refers to a disease or disorder resulting from an immune response against a self tissue or tissue component and includes a self antibody response or cell-mediated response. The autoimmune disease may be organ-specific, in which an autoimmune response is directed against a single tissue, such as Crohn's disease and ulcerative colitis, Type I diabetes mellitus, myasthenia gravis, vitiligo, Graves' disease, Hashimoto's disease, Addison's disease and autoimmune gastritis; and autoimmune hepatitis. Alternatively, the autoimmune disease may be non-organ specific autoimmune diseases, in which an autoimmune response is directed against a component present in several or many organs throughout the body. Such autoimmune diseases include, for example, rheumatoid disease, systemic lupus erythematosus, progressive systemic sclerosis and variants, polymyositis and dermatomyositis. Additional autoimmune diseases include, but are not limited to, pernicious anemia, autoimmune gastritis, primary biliary cirrhosis, autoimmune thrombocytopenia, Sjogren's syndrome, multiple sclerosis and psoriasis.

The phrase “inflammatory disease” as used herein, refers to a disease or disorder characterized or caused by inflammation. “Inflammation” refers to a local response to cellular injury that is marked by capillary dilatation, leukocytic infiltration, redness, heat, and pain that serves as a mechanism initiating the elimination of noxious agents and of damaged tissue. The site of inflammation includes the lungs, the pleura, a tendon, a lymph node or gland, the uvula, the vagina, the brain, the spinal cord, nasal and pharyngeal mucous membranes, a muscle, the skin, bone or bony tissue, a joint, the urinary bladder, the retina, the cervix of the uterus, the canthus, the intestinal tract, the vertebrae, the rectum, the anus, a bursa, a follicle, and the like. Such inflammatory diseases include, but are not limited to, fibrositis, inflammatory bowel disease, Crohn's disease, ulcerative colitis, pelvic inflammatory disease, acne, psoriasis, actinomycosis, dysentery, biliary cirrhosis, Lyme disease, heat rash, Stevens-Johnson syndrome, systemic lupus erythematosus, mumps, and blastomycosis.

The term “cancer” refers to any of various malignant neoplasms characterized by the proliferation of anaplastic cells that tend to invade surrounding tissue and metastasize to new body sites. Examples of different types of cancer include, but are not limited to, lung cancer, breast cancer, bladder cancer, thyroid cancer, liver cancer, pleural cancer, pancreatic cancer, ovarian cancer, cervical cancer, testicular cancer, colon cancer, anal cancer, colorectal cancer, bile duct cancer, gastrointestinal carcinoid tumors, esophageal cancer, gall bladder cancer, rectal cancer, appendix cancer, small intestine cancer, stomach (gastric) cancer, renal cancer, cancer of the central nervous system, skin cancer, choriocarcinomas; head and neck cancers; and osteogenic sarcomas, B-cell lymphoma, non-Hodgkin's lymphoma, Burkitt's lymphoma, fibrosarcoma, neuroblastoma, glioma, melanoma, monocytic leukemia, myelogenous leukemia, acute lymphocytic leukemia (ALL), osteosarcoma, acute myelocytic leukemia, malignant pleural mesothelioma and choriocarcinoma.

As used herein the term “anti-folate” refers to a compound which interferes with folate metabolism which is normally taken up into cells by the reduced folate carrier (RFC). Anti-folates may be classified into two groups—those that depend on folylpolyglutamate synthetase (FPGS) for activity (e.g. for intracellular retention), and those that do not. The phrase “folylpolyglutamate synthetase (FPGS) dependent antifolate” refers to the former. Examples of such anti-folates include, but are not limited to methotrexate, aminopeterin, tomudex (raltitrexed=ZD1694), 5,10-dideazatetrahydrofolic acid (DDATHF=lometrexol), Alimta (pemetrexed=LY231514=MTA), edatrexate, GW1843, and AG2034.

As mentioned, the method of this aspect of the present invention is effected by analyzing for a presence or absence of a splice variant of folylpolyglutamate synthetase (FPGS) or a polypeptide encoded thereby in a sample of the subject.

As used herein, the term “folylpolyglutamate synthetase” refers to the ATPase-type enzyme which is responsible for catalyzing the glutamylation of folates and anti-folates in the cell. In humans FPGS comprises a gene sequence as set forth in GeneBank Accession No. NM 004957—SEQ ID NO: 62.

As used herein, the phrase “splice variant” refers to a mRNA molecule (or cDNA produced therefrom) that arises from an alternative splicing event during RNA processing. Accordingly, the splice variant of the present invention is transcribed from a wild-type genomic DNA sequence (for example for humans that set forth in GeneBank Accession No. NM 004957) but has undergone splicing at a site which is alternative to that of a FPGS mRNA which encodes the wild-type polypeptide.

Alternative splicing results in the insertion or deletion of nucleic acids in the mRNA relative to the-wild type mRNA. In general, splice variants can generate both in-frame and frame-shift amino acid changes. Translation of a splice variant can result in a polypeptide with an amino acid sequence distinct from the wild type peptide resulting from conventional splicing, provided that the addition or deletion of nucleic acids are in frame. Translation of a splice variant could also result in a truncated polypeptide where a stop codon is introduced

The phrase “wild-type (wt) polypeptide” as used herein, refers to the polypeptide which comprises a sequence characteristic of most members of a species under natural conditions. For example, the wild-type human FPGS comprises an amino acid sequence as set forth in SEQ ID NO: 63 (Accession No: NM_{—}004957.4).

It will be appreciated that splice variants typically comprise mutations at positions of bridging between introns and exons.

According to one embodiment, the splice variants analyzed according to this aspect of the present invention comprise at least one RNA sequence variation in:

(i) a region bridging exon 2 and exon 3;

(ii) a region bridging exon 3 and exon 4;

(iii) a region bridging exon 6 and exon 7;

(iv) a region bridging exon 7 and exon 8;

(v) a region bridging exon 9 and exon 10;

(vi) a region bridging exon 10 and exon 11;

(vii) a region bridging exon 11 and exon 12; and/or

(viii) a region bridging exon 12 and exon 13.

Thus, embodiments of the present invention envisage analyzing the polynucleotide sequence of these bridging regions in the RNA or cDNA of the patient or analyzing the FPGS protein at amino acid sites corresponding to this bridging region.

For example, the splice variants of the present invention may comprise at least one of the following sequences (SEQ ID NOs: 47-51). Exemplary full-length FPGS polynucleotide sequences are set forth in SEQ ID NOs: 52-56.

According to a specific embodiment analyzing for the presence of splice variants is effected in vitro on a sample.

The term “sample” as used herein refers to a biological specimen obtained from the subject. Suitable samples for use in the present invention include, without limitation, whole blood, plasma, serum, red blood cells, saliva, urine, stool (i.e., feces), tears, any other bodily fluid, tissue samples (e.g., biopsy), and cellular extracts thereof (e.g., red blood cellular extract). In a preferred embodiment, the sample is obtained from peripheral blood or bone marrow.

Detection of the splice variants of the present invention can be effected using various methods known in the art, including RNA-based hybridization methods (e.g., Northern blot hybridization, RNA in situ hybridization and chip hybridization) reverse transcription-based detection methods (e.g., RT-PCR, quantitative RT-PCR, semi-quantitative RT-PCR, real-time RT-PCR, in situ RT-PCR, primer extension, mass spectroscopy, sequencing, sequencing by hybridization, LCR (LAR), Self-Sustained Synthetic Reaction (3SR/NASBA), Q-Beta (Qb) Replicase reaction, cycling probe reaction (CPR), a branched DNA analysis) and protein-based methods (e.g. Western blots and immunoprecipitation).

Total cellular RNA can be isolated from a biological sample using any suitable technique such as the single-step guanidinium-thiocyanate-phenol-chloroform method described in Chomczynski and Sacchi, Anal. Biochem. 162:156-159 (1987) or by using kits such as the Tri-Reagent kit (Sigma).

Following is a non-limiting list of RNA-based hybridization methods which can be used to detect the splice variants of the present invention.

Northern Blot analysis: This method involves the detection of a particular RNA in a mixture of RNAs. An RNA sample is denatured by treatment with an agent (e.g., formaldehyde) that prevents hydrogen bonding between base pairs, ensuring that all the RNA molecules have an unfolded, linear conformation. The individual RNA molecules are then separated according to size by gel electrophoresis and transferred to a nitrocellulose or a nylon-based membrane to which the denatured RNAs adhere. The membrane is then exposed to labeled DNA probes. Probes may be labeled using radio-isotopes or enzyme linked nucleotides. Typically, the DNA probe comprises a sequence which is not known to be altered in any of the splice variants of the present invention and is therefore capable of detecting both wt FPGS RNA and the splice variant forms thereof. Detection may be using autoradiography, colorimetric reaction or chemiluminescence. This method can be used for the determination of a size of the FPGS RNA. An aberrant size of the FPGS RNA which corresponds to a deletion of a particular exon (and/or inclusion of a particular intron) would suggest that the FPGS RNA is an aberrant splice variant.

RNA in situ hybridization stain: In this method DNA or RNA probes are attached to the RNA molecules present in the cells. Generally, the cells are first fixed to microscopic slides to preserve the cellular structure and to prevent the RNA molecules from being degraded and then are subjected to hybridization buffer containing the labeled probe. The hybridization buffer includes reagents such as formamide and salts (e.g., sodium chloride and sodium citrate) which enable specific hybridization of the DNA or RNA probes with their target mRNA molecules in situ while avoiding non-specific binding of probe. Those of skills in the art are capable of adjusting the hybridization conditions (i.e., temperature, concentration of salts and formamide and the like) to specific probes and types of cells. Following hybridization, any unbound probe is washed off and the slide is subjected to either a photographic emulsion which reveals signals generated using radio-labeled probes or to a colorimetric reaction which reveals signals generated using enzyme-linked labeled probes.

In situ RT-PCR stain: This method is described in Nuovo G J, et al. [Intracellular localization of polymerase chain reaction (PCR)-amplified hepatitis C cDNA. Am J Surg Pathol. 1993, 17: 683-90] and Komminoth P, et al. [Evaluation of methods for hepatitis C virus detection in archival liver biopsies. Comparison of histology, immunohistochemistry, in situ hybridization, reverse transcriptase polymerase chain reaction (RT-PCR) and in situ RT-PCR. Pathol Res Pract. 1994, 190: 1017-25]. Briefly, the RT-PCR reaction is performed on fixed cells by incorporating labeled nucleotides to the PCR reaction. The reaction is carried on using a specific in situ RT-PCR apparatus such as the laser-capture microdissection PixCell I LCM system available from Arcturus Engineering (Mountainview, Calif.).

Rnase protection assay: The technique can identify one or more RNA molecules of known sequence even at low total concentration. The extracted RNA is first mixed with antisense RNA or DNA probes that are complementary to the sequence or sequences of interest and the complementary strands are hybridized to form double-stranded RNA (or a DNA-RNA hybrid). The mixture is then exposed to ribonucleases that specifically cleave only single-stranded RNA but have no activity against double-stranded RNA. When the reaction runs to completion, susceptible RNA regions are degraded to very short oligomers or to individual nucleotides; the surviving RNA fragments are those that were complementary to the added antisense strand and thus contained the sequence of interest. The probes are prepared by cloning part of the gene of interest in a vector under the control of any of the following promoters, SP6, T7 or T3. These promotors are recognized by DNA dependent RNA polymerases originally characterized from bacteriophages. The probes produced maybe radioactive as they are prepared by in vitro transcription using radioactive UTPs. Uncomplemented DNA or RNA is cleaved off by nucleases. When the probe is a DNA molecule, Si nuclease is used; when the probe is RNA, any single-strand-specific ribonuclease can be used. Thus the surviving probe-mRNA complement is simply detected by autoradiography.

As mentioned, the splice variants of the present invention can be also detected using a reverse-transcription based method. Reverse-transcription utilizes RNA template, primers (specific or random), reverse transcriptase (e.g., MMLV-RT) and deoxyribonucleotides to form (i.e., synthesize) a complementary DNA (cDNA) molecule based on the RNA template sequence. Once synthesized, the single strand cDNA molecule or the double strand cDNA molecule (which is synthesized based on the single strand cDNA) can be used in various DNA based detection methods.

Following is a non-limiting list of methods which can directly or indirectly be used to detect the splice variants of the present invention from cDNA samples.

RT-PCR analysis—First, RNA molecules are purified from cells and converted into complementary DNA (cDNA) using a reverse transcriptase enzyme (such as an MMLV-RT) and primers such as oligo-dT, random hexamers, or gene-specific primers. Then by applying gene-specific primers and Taq DNA polymerase, a PCR amplification reaction is carried out in a PCR machine. Those of ordinary skill in the art are capable of selecting the length and sequence of the gene-specific primers and the PCR conditions (i.e., annealing temperatures, number of cycles, and the like) that are suitable for detecting specific RNA molecules. It will be appreciated that a semi-quantitative RT-PCR reaction can be employed, by adjusting the number of PCR cycles and comparing the amplification product to known controls. Primers used to detect splice variant mRNAs preferably hybridize to sequences flanking junction sites of deletions or to sequences flanking inserted sequences.

In situ RT-PCR stain—This method is described by: Nuovo, G. J. et al. (1993). Intracellular localization of polymerase chain reaction (PCR)-amplified hepatitis C cDNA. Am J Surg Pathol 17, 683-690); and Komminoth, P. et al. (1994) Evaluation of methods for hepatitis C virus detection in archival liver biopsies. Comparison of histology, immunohistochemistry, in situ hybridization, reverse transcriptase polymerase chain reaction (RT-PCR) and in situ RT-PCR. Pathol Res Pract 190, 1017-1025). Briefly, the RT-PCR reaction on fixed cells involves the incorporation of labeled nucleotides in the reaction. The reaction is effected using a specific in situ RT-PCR apparatus, such as the laser-capture microdissection PixCell II™ Laser Capture Microdissection (LCM) system available from Arcturus Engineering (Mountainview, Calif., USA).

Integrated systems—Another technique which may be used to analyze sequence alterations includes multicomponent integrated systems, which miniaturize and compartmentalize processes such as PCR and capillary electrophoresis reactions in a single functional device. An example of such a technique is disclosed in U.S. Pat. No. 5,589,136, which describes the integration of PCR amplification and capillary electrophoresis in chips.

Integrated systems are preferably employed along with microfluidic systems. These systems comprise a pattern of microchannels designed onto a glass, silicon, quartz, or plastic wafer included on a microchip. The movements of the samples are controlled by electric, electro-osmotic, or hydrostatic forces applied across different areas of the microchip, to create functional microscopic valves and pumps with no moving parts. Varying the voltage controls the liquid flow at intersections between the micro-machined channels and changes the liquid flow rate for pumping across different sections of the microchip.

When identifying sequence alterations, a microfluidic system may integrate nucleic acid amplification, microsequencing, capillary electrophoresis, and a detection method such as laser-induced fluorescence detection. In a first step, the DNA sample is amplified, preferably by PCR. The amplification product is then subjected to automated microsequencing reactions using ddNTPs (with specific fluorescence for each ddNTP) and the appropriate oligonucleotide microsequencing primers, which hybridize just upstream of the targeted polymorphic base. Once the extension at the 3′ end is completed, the primers are separated from the unincorporated fluorescent ddNTPs by capillary electrophoresis. The separation medium used in capillary electrophoresis can for example be polyacrylamide, polyethylene glycol, or dextran. The incorporated ddNTPs in the single-nucleotide primer extension products are identified by fluorescence detection. This microchip can be used to process 96 to 384 samples in parallel. It can use the typical four-color laser-induced fluorescence detection of ddNTPs.

It will be appreciated that when utilized along with automated equipment, the above-described detection methods can be both rapidly and easily used to screen multiple samples for the splice variants of the present invention.

Ligase Chain Reaction (LCR or LAR)—The ligase chain reaction [LCR; sometimes referred to as “Ligase Amplification Reaction” (LAR)] described by Barany, Proc. Natl. Acad. Sci., 88:189 (1991); Barany, PCR Methods and Applic., 1:5 (1991); and Wu and Wallace, Genomics 4:560 (1989) has developed into a well-recognized alternative method of amplifying nucleic acids. In LCR, four oligonucleotides, two adjacent oligonucleotides which uniquely hybridize to one strand of target DNA, and a complementary set of adjacent oligonucleotides, which hybridize to the opposite strand are mixed and DNA ligase is added to the mixture. Provided that there is complete complementarity at the junction, ligase will covalently link each set of hybridized molecules. Importantly, in LCR, two probes are ligated together only when they base-pair with sequences in the target sample, without gaps or mismatches. Repeated cycles of denaturation, and ligation amplify a short segment of DNA. LCR has also been used in combination with PCR to achieve enhanced detection of single-base changes. Segev, PCT Publication No. WO9001069 A1 (1990). However, because the four oligonucleotides used in this assay can pair to form two short ligatable fragments, there is the potential for the generation of target-independent background signal. The use of LCR for mutant screening is limited to the examination of specific nucleic acid positions.

Self-Sustained Synthetic Reaction (3SR/NASBA)—The self-sustained sequence replication reaction (3SR) (Guatelli et al., Proc. Natl. Acad. Sci., 87:1874-1878, 1990), with an erratum at Proc. Natl. Acad. Sci., 87:7797, 1990) is a transcription-based in vitro amplification system (Kwok et al., Proc. Natl. Acad. Sci., 86:1173-1177, 1989) that can exponentially amplify RNA sequences at a uniform temperature. The amplified RNA can then be utilized for mutation detection (Fahy et al., PCR Meth. Appl., 1:25-33, 1991). In this method, an oligonucleotide primer is used to add a phage RNA polymerase promoter to the 5′ end of the sequence of interest. In a cocktail of enzymes and substrates that includes a second primer, reverse transcriptase, RNase H, RNA polymerase and ribo- and deoxyribonucleoside triphosphates, the target sequence undergoes repeated rounds of transcription, cDNA synthesis and second-strand synthesis to amplify the area of interest. The use of 3SR to detect mutations is kinetically limited to screening small segments of DNA (e.g., 200-300 base pairs).

Q-Beta (Qβ) Replicase—In this method, a probe which recognizes the sequence of interest is attached to the replicatable RNA template for Qβ replicase. A previously identified major problem with false positives resulting from the replication of unhybridized probes has been addressed through use of a sequence-specific ligation step. However, available thermostable DNA ligases are not effective on this RNA substrate, so the ligation must be performed by T4 DNA ligase at low temperatures (37 degrees C.). This prevents the use of high temperature as a means of achieving specificity as in the LCR, the ligation event can be used to detect a mutation at the junction site, but not elsewhere.

Cycling probe reaction (CPR)—The cycling probe reaction (CPR) (Duck et al., BioTech., 9:142, 1990), uses a long chimeric oligonucleotide in which a central portion is made of RNA while the two termini are made of DNA. Hybridization of the probe to a target DNA and exposure to a thermostable RNase H causes the RNA portion to be digested. This destabilizes the remaining DNA portions of the duplex, releasing the remainder of the probe from the target DNA and allowing another probe molecule to repeat the process. The signal, in the form of cleaved probe molecules, accumulates at a linear rate. While the repeating process increases the signal, the RNA portion of the oligonucleotide is vulnerable to RNases that may carried through sample preparation.

Reverse dot-blot—This technique uses labeled sequence-specific oligonucleotide probes and unlabeled nucleic acid samples. Activated primary amine-conjugated oligonucleotides are covalently attached to carboxylated nylon membranes. After hybridization and washing, the labeled probe or a labeled fragment of the probe can be released using oligomer restriction, i.e., the digestion of the duplex hybrid with a restriction enzyme. Circular spots or lines are visualized colorimetrically after incubation with streptavidin horseradish peroxidase, followed by development using tetramethylbenzidine and hydrogen peroxide, or alternatively via chemiluminescence after incubation with avidin alkaline phosphatase conjugate and a luminous substrate susceptible to enzyme activation, such as CSPD, followed by exposure to x-ray film.

It will be appreciated that the splice variants of the present invention can be identified using a variety of sequence-based methods. Below is a summary of some exemplary sequence-based techniques.

Sequencing analysis—cDNA generated from the subject's RNA may be subjected to automated dideoxy terminator sequencing reactions using a dye-terminator (unlabeled primer and labeled di-deoxy nucleotides) or a dye-primer (labeled primers and unlabeled di-deoxy nucleotides) cycle sequencing protocols. For the dye-terminator reaction, a PCR reaction is performed using unlabeled PCR primers followed by a sequencing reaction in the presence of one of the primers, deoxynucleotides and labeled di-deoxy nucleotide mix. For the dye-primer reaction, a PCR reaction is performed using PCR primers conjugated to a universal or reverse primers (one at each direction) followed by a sequencing reaction in the presence of four separate mixes (correspond to the A, G, C, T nucleotides) each containing a labeled primer specific the universal or reverse sequence and the corresponding unlabeled di-deoxy nucleotides.

Microsequencing analysis—This analysis can be effected by conducting microsequencing reactions on specific regions e.g. the bridging regions between intron and exons which may be obtained by amplification reaction (PCR) such as mentioned hereinabove. Genomic or cDNA amplification products are then subjected to automated microsequencing reactions using ddNTPs (specific fluorescence for each ddNTP) and an appropriate oligonucleotide microsequencing primer which can hybridize just upstream of the alteration site of interest. Once specifically extended at the 3′ end by a DNA polymerase using a complementary fluorescent dideoxynucleotide analog (thermal cycling), the primer is precipitated to remove the unincorporated fluorescent ddNTPs. The reaction products in which fluorescent ddNTPs have been incorporated are then analyzed by electrophoresis on sequencing machines (e.g., ABI 377) to determine the identity of the incorporated base, thereby identifying the sequence alteration in the PDGFRα gene of the present invention.

It will be appreciated that the extended primer may also be analyzed by MALDI-TOF Mass Spectrometry. In this case, the base at the alteration site is identified by the mass added onto the microsequencing primer [see Haff and Smirnov, (1997) Nucleic Acids Res. 25(18):3749-50].

Solid phase microsequencing reactions which have been recently developed can be utilized as an alternative to the microsequencing approach described above. Solid phase microsequencing reactions employ oligonucleotide microsequencing primers or PCR-amplified products of the DNA fragment of interest which are immobilized. Immobilization can be carried out, for example, via an interaction between biotinylated DNA and streptavidin-coated microtitration wells or avidin-coated polystyrene particles.

In such solid phase microsequencing reactions, incorporated ddNTPs can either be radiolabeled [see Syvanen, (1994),] Clin Chim Acta 1994; 226(2):225-2369 or linked to fluorescein (see Livak and Hainer, (1994) Hum Mutat 1994; 3(4):379-385]. The detection of radiolabeled ddNTPs can be achieved through scintillation-based techniques. The detection of fluorescein-linked ddNTPs can be based on the binding of antifluorescein antibody conjugated with alkaline phosphatase, followed by incubation with a chromogenic substrate (such asp-nitrophenyl phosphate).

Other reporter-detection conjugates include: ddNTP linked to dinitrophenyl (DNP) and anti-DNP alkaline phosphatase conjugate [see Harju et al., (1993) Clin Chem 39:2282-2287]; and biotinylated ddNTP and horseradish peroxidase-conjugated streptavidin with o-phenylenediamine as a substrate (see WO 92/15712).

A diagnostic kit based on fluorescein-linked ddNTP with antifluorescein antibody conjugated with alkaline phosphatase is commercially available from GamidaGen Ltd (PRONTO).

Other modifications of the microsequencing protocol are described by Nyren et al. (1993) Anal Biochem 208(1):171-175 and Pastinen et al. (1997) Genome Research 7:606-614.

Methods for detecting the splice variants on the RNA level typically rely on the use of polynucleotide agents (i.e. probes) that are capable of hybridizing to the mutated form of the RNA. Methods for detecting the splice variant on the cDNA level typically rely on the use of polynucleotide agents (e.g. primers) that hybridize to the mutated sequences in the cDNA. As noted herein above, primers used to detect splice variant mRNAs may also hybridize to sequences flanking junction sites of deletions or to sequences flanking inserted sequences.

As used herein, the term “capable of hybridizing” refers to the ability to form a double strand molecule such as RNA:RNA, DNA:DNA and/or RNA:DNA molecules. According to one embodiment, the polynucleotide agents hybridize to the splice variants under physiological conditions.

“Physiological conditions” refer to the conditions present in cells, tissue or a whole organism or body. Preferably, the physiological conditions used by the present invention include a temperature between 34-40° C., more preferably, a temperature between 35-38° C., more preferably, a temperature between 36 and 37.5° C., most preferably, a temperature between 37 to 37.5° C.; salt concentrations (e.g., sodium chloride NaCl) between 0.8-1%, more preferably, about 0.9%; and/or pH values in the range of 6.5-8, more preferably, 6.5-7.5, most preferably, pH of 7-7.5.

Since the present inventors have shown that the splice variants may be devoid of exons (or parts thereof), the present invention contemplates using polynucleotide agents that hybridize to a polynucleotide that comprises the 3′ end of exon n directly linked to the 5′ end of exon n+2. Thus, for example, the present inventors have shown that the splice variants of the present invention are devoid of exon 3, exon 7, exon 10 and/or exon 12. Accordingly, the present invention contemplates positive identification by using polynucleotide agents that hybridize to a splice variant comprising the 3′ end of exon 2 directly linked to the 5′ end of exon 4 (e.g. SEQ ID NO: 41); the 3′ end of exon 6 directly linked to the 5′ end of exon 8 (e.g. SEQ ID NO: 43); the 3′ end of exon 9 directly linked to the 5′ end of exon 11 (e.g. SEQ ID NO: 13); and the 3′ end of exon 11 directly linked to the 5′ end of exon 13 (e.g. SEQ ID NO: 16). Negative identification may be effected by using polynucleotide agents that hybridize to a portion of the above identified exons. Alternatively, negative identification can be affected using polynucleotide agents that comprise the 3′ end of exon n directly linked to the 5′ end of exon n+1. Absence of hybridization would indicate that the RNA encoding the FPGS does not comprise this exon, or at least part thereof and can therefore be deemed a splice variant.

It will be appreciated that the splice variants of the present invention may also comprise introns. Positive identification of such introns may be effected by using polynucleotide agents that specifically hybridize with at least a portion thereof. Exemplary introns that may be retained in the splice variants of the present invention include, but are not limited to introns 1, 2, 10, 11 and 12.

As mentioned, the splice variants of the present invention may also be detected at the protein level.

Whilst chromatography and electrophoretic methods are preferably used to detect large variations in molecular weight, immunodetection assays such as ELISA and western blot analysis, immunohistochemistry and the like, which may be effected using antibodies that are capable of distinguishing between the wild-type form and splice variant form are preferably used to detect more subtle changes in molecular weight. Expression of the splice variants of the present invention can be determined using methods known in the arts.

Enzyme linked immunosorbent assay (ELISA): This method involves fixation of a sample (e.g., fixed cells or a proteinaceous solution) containing a protein substrate to a surface such as a well of a microtiter plate. A substrate specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western blot: This method involves separation of a substrate from other protein by means of an acrylamide gel followed by transfer of the substrate to a membrane (e.g., nylon or PVDF). Presence of the substrate is then detected by antibodies specific to the substrate, which are in turn detected by antibody binding reagents. Antibody binding reagents may be, for example, protein A, or other antibodies. Antibody binding reagents may be radiolabeled or enzyme linked as described hereinabove. Detection may be by autoradiography, colorimetric reaction or to chemiluminescence. This method allows both quantitation of an amount of substrate and determination of its identity by a relative position on the membrane which is indicative of a migration distance in the acrylamide gel during electrophoresis.

Radio-immunoassay (RIA): In one version, this method involves precipitation of the desired protein (i.e., the substrate) with a specific antibody and radiolabeled antibody binding protein (e.g., protein A labeled with I¹²⁵) immobilized on a precipitable carrier such as agarose beads. The number of counts in the precipitated pellet is proportional to the amount of substrate.

In an alternate version of the RIA, a labeled substrate and an unlabelled antibody binding protein are employed. A sample containing an unknown amount of substrate is added in varying amounts. The decrease in precipitated counts from the labeled substrate is proportional to the amount of substrate in the added sample.

Fluorescence activated cell sorting (FACS): This method involves detection of a substrate in situ in cells by substrate specific antibodies. The substrate specific antibodies are linked to fluorophores. Detection is by means of a cell sorting machine which reads the wavelength of light emitted from each cell as it passes through a light beam. This method may employ two or more antibodies simultaneously.

Immunohistochemical analysis: This method involves detection of a substrate in situ in fixed cells by substrate specific antibodies. The substrate specific antibodies may be enzyme linked or linked to fluorophores. Detection is by microscopy and subjective or automatic evaluation. If enzyme linked antibodies are employed, a colorimetric reaction may be required. It will be appreciated that immunohistochemistry is often followed by counterstaining of the cell nuclei using for example Hematoxyline or Giemsa stain.

For positive identification, the antibody may specifically bind at least one epitope which is present in a splice variant, but absent in the wild-type form. Conversely, for negative identification, the antibody may specifically bind at least one epitope which is present in a wild-type form, but absent in the splice variant.

According to one embodiment, the antibody has at least 10 fold higher affinity for the splice variant polypeptide than the wild-type polypeptide. According to another embodiment, the antibody has at least 50 fold higher affinity for the splice variant polypeptide than the wild-type polypeptide.

As used herein, the term “epitope” refers to any antigenic determinant on an antigen to which the paratope of an antibody binds.

Epitopic determinants usually consist of chemically active surface groupings of molecules such as amino acids or carbohydrate side chains and usually have specific three dimensional structural characteristics, as well as specific charge characteristics.

The term “antibody” as used in this invention includes intact molecules as well as functional fragments thereof, such as Fab, F(ab′)2, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5 S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The probes, primers and/or antibodies (i.e. detecting agents) which are capable of detecting the splice variants of the present invention may be provided in kits. The kits may further include a DNA polymerase enzyme, such as a thermostable DNA polymerase, a reverse transcriptase enzyme, a mixture of dNTPs, a concentrated polymerase chain reaction buffer and a concentrated reverse transcription buffer. The detecting agents can include nucleotide analogs and/or a labeling moiety, e.g., directly detectable moiety such as a fluorophore (fluorochrome) or a radioactive isotope, or indirectly detectable moiety, such as a member of a binding pair, such as biotin, or an enzyme capable of catalyzing a non-soluble colorimetric or luminometric reaction. The kit may also comprise at least one precast gel for executing RT-PCR or Western Blot analysis. In addition, the kit may further include at least one container containing reagents for detection of electrophoresed nucleic acids. Such reagents include those which directly detect nucleic acids, such as fluorescent intercalating agent or silver staining reagents, or those reagents directed at detecting labeled nucleic acids, such as, but not limited to, ECL reagents. The kit preferably includes a notice associated therewith in a form prescribed by a governmental agency regulating the manufacture, use or sale of diagnostic kits. Detailed instructions for use, storage and trouble shooting may also be provided with the kit.

As mentioned, the present inventors have shown that a presence of the splice mutants of the present invention predict a patient's responsiveness to anti-folate therapy. Accordingly, if a patient is found to be positive for the presence of a FPGS splice variant, selection of an alternative therapy may prove to be more effective for treating the patient.

According to one embodiment, the alternative therapy may comprise an anti-folate that is not dependent on FPGS activity. Examples of such anti-folates include, but are not limited to plevitrexed (BGC 9331=ZD9331), piritrexim, neutrexin (trimetrexate), PT523 and AG337 (Nolatrexed, Thymitaq).

Since the present inventors identified novel FPGS variants, the present invention further contemplates isolated polynucleotides encoding FPGS polypeptides comprising a nucleic acid sequence at least 60% at least 65% at least 70% at least 75% at least 80% at least 85% at least 86% at least 87% at least 88% at least 89% at least 90% at least 91% at least 92% at least 93% at least 94% at least 95% at least 96% at least 97% at least 98% at least 99% or 100% homologous to at least one of the sequences selected from the group consisting of SEQ ID NOs: 47-51, as determined by BlastP of the National Center of Biotechnology Information [(NCBI) wwwdotncbidotnihdotgov/BLAST/] using default parameters.

As used herein the phrase “an isolated polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is at least partially complementary and at least partially genomic. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

According to one embodiment of this aspect of the present invention the nucleic acid sequence is as set forth in SEQ ID NOs: 52-56.

The isolated polynucleotides of the present invention can be qualified using hybridization assays. Moderate to stringent hybridization conditions are characterized by a hybridization solution such as containing 10% dextrane sulfate, 1 M NaCl, 1% SDS and 5×10⁶ cpm ³²P labeled probe, at 65° C., with a final wash solution of 0.2×SSC and 0.1% SDS and final wash at 65° C. and whereas moderate hybridization is effected using a hybridization solution containing 10% dextrane sulfate, 1 M NaCl, 1% SDS and 5×10⁶ cpm ³²P labeled probe, at 65° C., with a final wash solution of 1×SSC and 0.1% SDS and final wash at 50° C.

Thus, the present invention encompasses nucleic acid sequences described hereinabove; fragments thereof, sequences hybridizable therewith, sequences homologous thereto, sequences encoding similar polypeptides with different codon usage, altered sequences characterized by mutations, such as deletion, insertion or substitution of one or more nucleotides, either naturally occurring or man induced, either randomly or in a targeted fashion.

Since the polynucleotide sequences of the present invention encode previously unidentified polypeptides, the present invention also encompasses novel polypeptides of FPGS or portions thereof, which are encoded by the isolated polynucleotide and respective nucleic acid fragments thereof described hereinabove.

Thus, the present invention also encompasses polypeptides encoded by the novel FPGS nucleic acid sequences of the present invention. Examples of amino acid sequences of these novel polypeptides are set forth in SEQ ID NO: 57-61.

The present invention also encompasses homologs of these polypeptides, such homologs can be at least 60% at least 65% at least 70% at least 75% at least 80% at least 85% at least 86% at least 87% at least 88% at least 89% at least 90% at least 91% at least 92% at least 93% at least 94% at least 95% at least 96% at least 97% at least 98% at least 99% or more say 100% homologous to SEQ ID NOs: 57-61.

Finally, the present invention also encompasses fragments of the above described polypeptides and polypeptides having mutations, such as deletion, insertion or substitution of one or more amino acids, either naturally occurring or man induced, either randomly or in a targeted fashion.

It will be appreciated that the polypeptides of the present invention may be degradation products, synthetic peptides or recombinant peptides as well as peptidomimetics, typically, synthetic peptides and peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2—NH, CH2—S, CH2—S═O, O═C—NH, CH2—O, CH2—CH2, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated bonds (—N(CH3)—CO—), ester bonds (—C(R)H—C—O—O—C(R)—N—), ketomethylen bonds (—CO—CH2—), α-aza bonds (—NH—N(R)—CO—), wherein R is any alkyl, e.g., methyl, carba bonds (—CH2—NH—), hydroxyethylene bonds (—CH(OH)—CH2—), thioamide bonds (—CS—NH—), olefinic double bonds (—CH═CH—), retro amide bonds (—NH—CO—), peptide derivatives (—N(R)—CH2—CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted for synthetic non-natural acid such as Phenylglycine, TIC, naphthylelanine (Nol), ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables 1 and 2 below list naturally occurring amino acids (Table 1) and non-conventional or modified amino acids (Table 2) which can be used with the present invention.

TABLE 1

| | Three-Letter | |
|---|---|---|
| Amino Acid | Abbreviation | One-letter Symbol |
| alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| glycine | Gly | G |
| Histidine | His | H |
| isoleucine | Iie | I |
| leucine | Leu | L |
| Lysine | Lys | K |
| Methionine | Met | M |
| phenylalanine | Phe | F |
| Proline | Pro | P |
| Serine | Ser | S |
| Threonine | Thr | T |
| tryptophan | Trp | W |
| tyrosine | Tyr | Y |
| Valine | Val | V |
| Any amino acid as above | Xaa | X |

TABLE 2

| Non-conventional amino | | | |
|---|---|---|---|
| acid | Code | Non-conventional amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgin |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3-diphenylpropyl)glycine | Nbhe |
| D-N-methylleucine | Dnmleu | N-(3-indolylyethyl) glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomo phenylalanine | Mhphe |
| L-α-methylisoleucine | Mile | N-(2-methylthioethyl)glycine | Nmet |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | mser | L-α-methylthreonine | Mthr |
| L-α-methylvaline | Mtrp | L-α-methyltyrosine | Mtyr |
| L-α-methylleucine | Mval | L-N-methylhomophenylalanine | Nmhphe |
| | Nnbhm | | |
| N—(N-(2,2-diphenylethyl) | | N—(N-(3,3-diphenylpropyl) | |
| carbamylmethyl-glycine | Nnbhm | carbamylmethyl(1)glycine | Nnbhe |
| 1-carboxy-1-(2,2-diphenyl | Nmbc | | |
| ethylamino)cyclopropane | | | |

The polypeptides of the present may be in a linear form, although it will be appreciated that in cases where cyclicization does not severely interfere with peptide characteristics, cyclic forms of the polypeptide are also contemplated.

The peptides of present invention can be biochemically synthesized such as by using standard solid phase techniques. These methods include exclusive solid phase synthesis, partial solid phase synthesis methods, fragment condensation, classical solution synthesis. These methods are preferably used when the peptide is relatively short (i.e., 10 kDa) and/or when it cannot be produced by recombinant techniques (i.e., not encoded by a nucleic acid sequence) and therefore involves different chemistry.

Solid phase peptide synthesis procedures are well known in the art and further described by John Morrow Stewart and Janis Dillaha Young, Solid Phase Peptide Syntheses (2nd Ed., Pierce Chemical Company, 1984).

Synthetic polypeptides can be purified by preparative high performance liquid chromatography [Creighton T. (1983) Proteins, structures and molecular principles. WH Freeman and Co. N.Y.] and the composition of which can be confirmed via amino acid sequencing.

In cases where large amounts of the polypeptides of the present invention are desired, the polypeptides can be generated using recombinant techniques using a nucleic acid expression construct (further described hereinbelow). Recombinant production of polypeptides is described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463 and further below.

It is expected that during the life of a patent maturing from this application many relevant anti-folates will be developed and the scope of the term anti-folate is intended to include all such new technologies a priori.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Aberrant Splicing of Folylpolyglutamate Synthetase as a Novel Mechanism of Antifolate Resistance in Leukemia

Materials and Methods

Tissue Culture: The human T-cell lymphocytic leukemia lines CCRF-CEM and Molt-4 as well as their antifolate-resistant sublines CEM-MTX^{R5}P, CEM-MTX^{R10}, CEM-7OH MTX and Molt-4-7OH MTX were maintained in RPMI-1640 medium (GIBCO) containing 2.3 μM folic acid supplemented with 10% fetal bovine serum, 2 mM glutamine, 100 units/ml penicillin G and 100 μg/ml streptomycin sulfate (Biological Industries, Beth-Haemek, Israel). The MTX-resistant sublines MTX^{R5}P and MTX^{R10} were established by exposure of parental CCRF-CEM cells to 6 cycles each of 24 hr pulse of 5 or 10 μM MTX, respectively (Sigma) as follows: after an initial 24 hr pulse exposure of parental CCRF-CEM cells to 5 or 10 μM MTX at 37° C., cells were washed once with drug-free growth medium, adjusted to 3×10⁵ cells/ml and allowed to grow for 1-2 weeks. Thereafter, a maintenance treatment of 24 hr pulses of MTX was performed once a month. CEM-7OH MTX and Molt-4-7OH MTX were established and maintained as previously described [Fotoohi K, et al., Blood. 2004; 104:4194-4201].

Antifolate growth inhibition assay: Parental cells and their antifolate-resistant sublines as well as FPGS-transfected cells were first grown in antifolate-free growth medium for 5-7 cell doublings. Thereafter, cells were seeded in 96-well plates (3×10⁴/well) in growth medium (100 μl/well) containing various concentrations of the different antifolates (e.g. MTX, ZD1694 and ZD9331). After 3 days of incubation at 37° C., viable cell numbers were determined using the Cell Proliferation Kit (XTT) (Biological Industries). Percent inhibition of cell growth was calculated relative to untreated controls. Results presented are means of at least three independent experiments.

[³H]MTX transport assay: [³H]MTX influx rates were determined as previously described [Jansen Get al., J Biol Chem. 1990; 265:18272-18277]. Briefly, cells (2×10⁷) were washed with ice-cold HBS (20 mM HEPES, 140 mM NaCl, 5 mM KCl, 2 mM MgCl₂, and 5 mM glucose, at pH 7.4) and incubated at 37° C. for 3 min in HBS containing 2 μM [3′,5′,7-³H]-Methotrexate (0.555 TBq/mmol; 26.7 Ci/mmol; Moravek Biochemicals, Brea, Calif.). Transport controls contained a 500-fold excess (1 mM) of unlabeled MTX. Transport was stopped by the addition of 10 ml of ice-cold HBS. Then, the cell suspension was centrifuged, washed with ice-cold HBS and suspended in water for scintillation counting.

FPGS activity assay: The catalytic activity of FPGS was determined as described previously [Li W W et al., Cancer Res. 1992; 52:3908-3913]. In short, frozen cell pellets (2×10⁷ cells) were suspended in 0.4 ml extraction buffer containing: 50 mM Tris-HCl, 20 mM KCl, 10 mM MgCl₂ and 5 mM dithiotreitol, at pH 7.5. Crude cell extracts were prepared by sonication (MSE Soniprep, amplitude 6, 3×5 sec with 30 sec intervals, at 4° C.) followed by centrifugation at 12,000×g for 15 min at 4° C. The activity assay mixture consisted of: 200 μg protein, 4 mM [2,3-³H]-L-glutamic acid (NEN Life Science Products, Boston, Mass.) and 250 μM MTX in a buffer containing: 0.5 M Tris-HCl, 50 mM ATP, 100 mM MgCl₂, 100 mM KCl, and 5 mM dithiotreitol at a pH of 8.5. Following 2 h incubation at 37° C., the reaction was terminated by adding 1 ml of an ice-cold solution containing 5 mM unlabeled L-glutamic acid. Sep-Pak C₁₈ cartridges (Millipore, Waters Associates, Etten-Leur, The Netherlands) were used in order to eliminate free [³H]-L-glutamate.

Semi-quantitative RT-PCR Analysis of Various Genes: Cells (1×10⁷) from the mid-log phase of growth were harvested by centrifugation, washed with phosphate buffered saline (PBS) and total RNA was isolated using the Tri-Reagent kit according to the instructions of the manufacturer (Sigma) and treated with DNase (Promega). A portion of total RNA (12 μg in a total volume of 50 μl) was reverse transcribed using M-MLV (400 units, Promega) in a reaction buffer containing random hexamer primers (Promega), dNTPs (Larova,) and a ribonuclease inhibitor Rnasin (TaKaRa). PCR was performed using 10 pmols of each primer (Tables 3, 4 and 5, herein below) in 2×ReddyMix PCR master mix reaction buffer according to the instructions of the manufacturer (ABgene). Then, the PCR products were resolved on 1% agarose gels containing ethidium bromide.

TABLE 3
Primer pairs use to screen the entire FPGS gene

| | Position | | Position | |
|---|---|---|---|---|
| | on | Length | on | Length |
| Primer | cDNA | (bp) | gDNA | (bp) |
| EX1-up | 108-125 | 263 | Exon 1 | 1570 |
| ′5-CGCGGCATAACGACCCA | | | | |
| G-′3 | | | | |
| (SEQ ID NO: 1) | | | | |
| EX3-dw | 351-370 | | Exon 3 | |
| ′5-TTCCCCTTCGTCCCAGT | | | | |
| GAC-′3 | | | | |
| (SEQ ID NO: 2) | | | | |
| EX3-up | 332-349 | 567 | Exon 3 | 4071 |
| ′5-CCGGCTGAACATCATCC | | | | |
| A-′3 | | | | |
| (SEQ ID NO: 3) | | | | |
| EX10-dw | 878-898 | | Exon 19 | |
| ′5-AGCATCGGACACAGGTA | | | | |
| TAGA-′3 | | | | |
| (SEQ ID NO: 4) | | | | |
| EX8-up | 712-730 | 594 | Exon 8 | 2452 |
| ′5-TCTCCTCTCTTGGCATC | | | | |
| GA-′3 | | | | |
| (SEQ ID NO: 5) | | | | |
| EX13-dw | 1288-1306 | | Exon 13 | |
| ′5-CGGTCCCCGGTAGCATT | | | | |
| GA-′3 | | | | |
| (SEQ ID NO: 6) | | | | |
| EX11-up | 1033-1051 | 460 | Exon 11 | 4471 |
| ′5-CAAAGGCATCCAGGCCA | | | | |
| GG-′3 | | | | |
| (SEQ ID NO: 7) | | | | |
| EX15-dw | 1473-1492 | | Exon 15 | |
| ′5-TGCTCTTCGTCCAGGTG | | | | |
| GTT-′3 | | | | |
| (SEQ ID NO: 8) | | | | |
| EX15-up | 1414-1435 | 467 | Exon 15 | 467 |
| ′5-TTCACAGTGACACTGGA | | | | |
| CCA-′3 | | | | |
| (SEQ ID NO: 9) | | | | |
| EX15b-dw | 1862-1882 | | Exon 15 | |
| ′5-AGTATGTAAGTTCATGG | | | | |
| GGAG-′3 | | | | |
| (SEQ ID NO: 10) | | | | |

TABLE 4
Diagnostic PCR primers use to identify splicing
alteration in the FPGS gene

| | | Length |
|---|---|---|
| Primer | Position on gDNA | (bp) |
| #INT10 | Intron 10 | 1368 |
| ′5-GAGTGGGCAGCTGAGTGGG-′3 | | |
| (SEQ ID NO: 11) | | |
| #ex9-10 | ′3 of exon 9 + | 449 |
| ′5-GCCCAGCAGATCTCATGTCC-′3 | ′5 of exon 10 | |
| (SEQ ID NO: 12) | | |
| #ex9-11 | | |
| 5′-CAGATCTCATGCTGGGGAGC-′3 | ′3 of exon 9 + | 294 |
| (SEQ ID NO: 13) | ′5 of exon 11 | |
| #ex10in10 | ′3 of exon 10 + | 393 |
| 5′-AGGACCGCCATGGTGAGTG-′3 | ′5 of intron 10 | |
| (SEQ ID NO: 14) | | |
| *ex11-12 | ′3 of exon 11 + | 387 |
| 5′-GCTCGGGCTTCGGAACAC-′3 | ′5 of exon 12 | |
| (SEQ ID NO: 15) | | |
| *ex 11-13 | ′3 of exon 11 + | 245 |
| 5′-CCCACATGCGGCTCGTG-′3 | ′5 of exon 13 | |
| (SEQ ID NO: 16) | | |

#Forward primer used with the reverse primer EX13-dw from Table 3.
*Forward primer used with the reverse primer EX15-dw from Table 3.

TABLE 5
PCR primer pairs used to examine the splicing of various genes

| | | | Position | | Position | |
|---|---|---|---|---|---|---|
| Gene | Accession | Primer | on cDNA | Length | on gDNA | Length |
| B-actin | CR624852 | β-actin-up | 160-179 | 576 | Exon 2 | 1151 |
| | | ′5-CCGTCTTCCCCTCCATCGTG-′3 | | | | |
| | | (SEQ ID NO: 17) | | | | |
| | | β-actin-dw | 715-735 | | Exon 4 | |
| | | ′5-GGGCGACGTAGCACAGCTTCT-′3 | | | | |
| | | (SEQ ID NO: 18) | | | | |
| RNA | NM_015972 | RNA-up | 94-114 | 310 | Exon 1 | 1176 |
| polymerase ID | | ′5-TGGAAGAGGATCAGGAGCTGG-′3 | | | | |
| | | (SEQ ID NO: 19) | | | | |
| | | RNA-dw | 383-404 | | Exon 2 | |
| | | ′5-CATGAGCTCATTCAGGCCTCTC-′3 | | | | |
| | | (SEQ ID NO: 20) | | | | |
| MRP1 | NM_004996 | MRP-EX23 | 3776-3794 | 198 | Exon 23 | 1092 |
| | | ′5-GTGCGGCTGGAGTGTGTGG-′3 | | | | |
| | | (SEQ ID NO: 21) | | | | |
| | | MRP-EX24 | 3954-3973 | | Exon 24 | |
| | | ′5-CTCCTTGAGCCTCTCCACGG-′3 | | | | |
| | | (SEQ ID NO: 22) | | | | |
| TS | NM_001071 | TS-U5 | 879-898 | 142 | Exon 6 | 1550 |
| | | ′5-CCTGAATCACATCGAGCCAC-′3 | | | | |
| | | (SEQ ID NO: 23) | | | | |
| | | TS-D4 | 1001-1020 | | Exon 7 | |
| | | ′5-TGGATGCGGATTGTACCCTT-′3 | | | | |
| | | (SEQ ID NO: 24) | | | | |
| DHFR | NM_00791 | DHFR-EX1 | 502-521 | 115 | Exon 1 | 460 |
| | | ′5-TCGCTAAACTGCATCGTCGC-′3 | | | | |
| | | (SEQ ID NO: 25) | | | | |
| | | DHFR-D1 | 597-616 | | Exon 2 | |
| | | ′5-AGGTTGTGGTCATTCTCTGG-′3 | | | | |
| | | (SEQ ID NO: 26) | | | | |
| RFC | U19720.1 | RFC2-up | 581-598 | 576 | Exon 3 | 993 |
| | | ′5-GGGCGTGTTCACCAGCTC-′3 | | | | |
| | | (SEQ ID NO: 27) | | | | |
| | | RFC2-dw | 1138-1156 | | Exon 4 | |
| | | ′5-GCCAGAAGGAAGACCAGCC-′3 | | | | |
| | | (SEQ ID NO: 28) | | | | |
| PDGF | M63193 | TP02-up | 290-309 | 350 | Exon 2 | 1030 |
| | | ′5-GCTTCGTGGCCGCTGTGGTG-′3 | | | | |
| | | (SEQ ID NO: 29) | | | | |
| | | TP02-dw | 620-639 | | Exon 4 | |
| | | ′5-CTGCTCTGGGCTCTGGATGA-′3 | | | | |
| | | (SEQ ID NO: 30) | | | | |
| GAPDH | NM_002046 | GAP-up | 440-457 | 541 | Exon 6 | 826 |
| | | ′5-AGGGGGGAGCCAAAAGGG-′3 | | | | |
| | | (SEQ ID NO: 31) | | | | |
| | | GAP-up | 960-980 | | Exon 8 | |
| | | ′5-GAGGAGTGGGTGTCGCTGTTG-′3 | | | | |
| | | (SEQ ID NO: 32) | | | | |
| TNFα | NM_000594 | TNFaG-up | 402-420 | 301 | Exon 3 | 602 |
| | | ′5-GATCATCTTCTCGAACCCCG-′3 | | | | |
| | | (SEQ ID NO: 33) | | | | |
| | | TNFaG-dw | 685-702 | | Exon 4 | |
| | | ′5-TGGCAGGGGCTCTTGATG-′3 | | | | |
| | | (SEQ ID NO: 34) | | | | |
| UBTF | NM_014233 | UBTF-up | 131-151 | 356 | Exon 2 | 991 |
| | | ′5-TGGTCCCAGGAAGACATGCTG-′3 | | | | |
| | | (SEQ ID NO: 35) | | | | |
| | | UBTF-dw | 466-486 | | Exon 4 | |
| | | ′5-AGGTTGCTCATCTCAGGGTGG-′3 | | | | |
| | | (SEQ ID NO: 36) | | | | |
| Plexin A1 | NM_032242 | Plex-up | 3431-3451 | 362 | Exon 17 | 847 |
| | | ′5-AGCTCCCCACTCATCCTCAAG-′3 | | | | |
| | | (SEQ ID NO: 37) | | | | |
| | | Plex-dw | 3774-3792 | | Exon 19 | |
| | | ′5-TCCATCTGGAGCTGCAGCC-′3 | | | | |
| | | (SEQ ID NO: 38) | | | | |

DNA Sequencing: PCR products were first purified using the Wizard SV Gel and PCR Clean-up kit according to the instructions of the manufacturer (Promega). Then, DNA sequencing was performed at HyLabs laboratories (Rehovot, Israel) using BigDye Terminator Cycle Sequencing Kit from ABI.

Expression Vectors: pcDNA3/hFPGS harboring the full-length human FPGS cDNA was provided. In order to construct a vector containing the FPGS gene lacking exon 10, the FPGS cDNA was PCR amplified with PfuTurbo DNA polymerase (Stratagene) using the following primers: Nhe/FPGS ′5-tatagctagccaccatggagtaccaggatg-′3 (SEQ ID NO: 39) and FPGS/EcoRI ′5-ttaagaattcgccttggctactgggac-′3 (SEQ ID NO: 40); the PCR product was then digested with Nhe I and Hind III (Fermentas) and cloned into pIRES2-EGFP (Clontech) upstream of the IRES element. Deletion of exon 10 was performed using the Site Directed Mutagenesis kit (Stratagene) and the new vector was termed pIRES/FPGSΔexon 10.

Stable Transfections with hFPGS Expression Constructs: Exponentially growing cells (i.e. CCRF-CEM and)CEM-MTX^{R10}) were harvested by centrifugation and stably transfected by electroporation (1000 μF, 234V) with 10 μg of the vectors pcDNA3.1, pIRES2-EGFP, pcDNA3/hFPGS or pIRES/FPGSΔexon10. After 24 h of growth at 37° C., cells were exposed to 450 to 600 μg/ml active G-418 and single clones were then picked and expanded. Stable transfectants obtained after 4 weeks of G-418 selection were analyzed for FPGS mRNA levels by Northern blots as detailed below and used for further analyses.

Northern Blot Analysis: Aliquots (30 μg) of total RNA isolated as described above from parental CCRF-CEM cells and their various antifolate-resistant sublines and transfectants were denatured at 68° C. for 10 min in a MOPS-formamide buffer and fractionated by electrophoresis on 1.2% agarose gels containing formaldehyde. Resolved RNA was then capillary blotted onto Zeta-Probe^{R}-GT nylon membrane (Bio-Rad) and immobilized onto the nylon membrane by UV cross-linking. The nylon membrane was stained with methylene blue in order to verify equal loading and transfer. A 567 bp FPGS cDNA probe prepared by PCR with primers EX3-up (SEQ I NO: 3) and EX10-dw (SEQ ID NO: 4) (Table 3, herein above) was gel-purified using the Wizard SV Gel and PCR Clean-up kit (Promega) and [³²P]labeled using Random Primer DNA Labeling Mix (Biological Industries, Beth Haemek, Israel). The blot was then hybridized over-night with the above probe in glass cylinders in EZ-Hybridization Solution (Biological Industries, Beth Haemek, Israel) at 0.1 ml buffer/cm² at 68° C. Following hybridization, the membrane was washed under high stringency conditions with a final wash in a solution of 0.1×SSC/0.1% SDS at 65° C. for 30 min. The membrane was then visualized by phosphorimaging and the intensity of the FPGS transcript was estimated by scanning densitometry and normalization to the 18S ribosomal RNA band.

Leukemia Patient Specimens: Analysis of FPGS splicing defects was performed on stored RNA samples previously obtained from adult ALL patients that were treated according to the UKALL12/ECOG 2993 protocol [Goldstone A H et al., Blood. 2008; 111:1827-1833]. 13 RNA samples were studied derived from 8 ALL patients, five of which were matched samples both at the time of first diagnosis and relapse, as well as 3 unpaired diagnosis samples. The samples had been originally used (i.e. irrespective to the current study) for RNA extraction; leukocytes were isolated from either peripheral blood or bone marrow by a standard Ficoll-Hypaque density centrifugation and total RNA was purified using the Tri-Reagent kit according to the instructions of the manufacturer (Sigma). Aliquots of RNA stored at −80° C. were reverse transcribed as described above. The entire FPGS gene was amplified by PCR, screened for either intron retention and/or exon skipping using primers from Tables 3 and 4 as well as from Liani et. al., Int J Cancer. 2003; 103:587-599.

Results

Resistance to Polyglutamatable Antifolates in MTX^{R5}P Leukemia Cells is Associated with a Marked Loss of FPGS Activity

In order for polyglutamatable antifolates to exert their cytotoxic activity, they must first undergo polyglutamylation catalyzed by FPGS. Hence, loss of FPGS activity results in resistance to polyglutamylation-dependent antifolates. Consistently, MTX^{R5}P leukemia cells were found here to display less than 1% of FPGS activity relative to their parental CCRF-CEM cells; this profound loss of FPGS activity was comparable to that of another FPGS-deficient cell line CEM/MTX^{R10} that was established (Table 6, herein below).

TABLE 6
Values for Growth Inhibition, MTX Influx and FPGS Activity Assays

| | | | | MTX | FPGS |
|---|---|---|---|---|---|
| Antifolate | ZD1964 | ZD9331 | MTX | Influx | Activity |
| Cell line | IC₅₀ [nM] | IC₅₀ [nM] | IC₅₀ [nM] | (%) | (%) |
| CCRF- | 6.06 ± 0.42 | 28.6 ± 2.3 | 25.7 ± 2.1 | 100 | 100 |
| CEM WT | | | | | |
| CEM- | >300,000 | 13.9 ± 1.7 | 23.1 ± 2 | 50 ± 5 | 0.8 ± 0.2 |
| MTX^{R5}P | | | | | |
| CEM- | 110.1 ± 9.8 | 40.8 ± 4.1 | 28.2 ± 2.9 | 91 + 6 | 1.2 + 0.4 |
| MTX^{R10} | | | | | |

Moreover, RFC (SLC19A1), the predominant transporter mediating the uptake of reduced folates and antifolates, retained as much as 50% of its [³H]MTX transport activity, when compared to parental cells (Table 6). Hence, a substantial preservation of RFC activity was observed in MTX^{R5}P cells. The major loss of FPGS activity in MTX^{R5}P cells resulted in a 50,000-fold resistance to raltitrexed (Tomudex; ZD1694), a polyglutamylation-dependent antifolate that exerts its cytotoxic activity via potent inhibition of thymidylate synthase (TS; Table 6). In contrast, when compared to their parental cells, MTX^{R5}P cells were 2-fold more sensitive to the polyglutamylation-independent antifolate, ZD9331 that also potently inhibits TS activity. Thus, these results suggest that the mechanism underlying antifolate-resistance in MTX^{R5}P cells is loss of FPGS activity.

MTX^{R5}P Cells Display Retention of Introns in the mRNA of FPGS and Other Genes

To explore the molecular basis of the loss of FPGS function in MTX^{R5}P cells, the entire FPGS coding region was screened for inactivating mutations using overlapping RT-PCR primers (Table 3, herein above). While screening for mutations in MTX^{R5}P cells, the present inventors surprisingly obtained markedly longer FPGS PCR fragments than expected (FIG. 1A, lanes 2, 4), whereas parental cells showed a PCR product with the expected size or no product at all (FIG. 1A, lanes 1 and 3 respectively). DNA sequencing of these long PCR products from MTX^{R5}P cells revealed that introns 1, 2, 10, 11 and 12 were retained in the FPGS mRNA (FIG. 1A; introns 1,2 and 10-12, lanes 2 and 4, respectively). These findings were indicative of defective splicing which could constitute the molecular basis for the loss of FPGS activity in these antifolate-resistant cells. To examine the status of global splicing in MTX^{R5}P cells, the present inventors undertook an RT-PCR analysis on an assortment of genes using primers positioned within neighboring exons, thereby allowing for the formation of two distinct fragments that are diagnostic of intron retention. The different primers used for this PCR analysis and the various possible product sizes are depicted in Table 5, herein above, and the PCR products are shown in FIGS. 1B-C. Interestingly, while essential genes such as RNA polymerase ID and β-actin exhibit normal size PCR products (FIG. 1B), a variety of other genes showed defective splicing; the latter genes included upstream binding transcription factor, UBTF; tumor necrosis factor α, TNFα; glyceraldehyde-3-phosphate dehydrogenase, GAPDH; platelet-derived growth factor, PDGF; RFC, as well as plexin A1. Therefore, at least 50% of the transcripts encoded by these genes contained unspliced introns, thereby corroborating the prominent splicing defect affecting various genes in MTX^{R5}P cells (FIG. 1C). To verify the presence and identity of the unspliced introns, DNA sequencing of all aberrant RT-PCR products was undertaken. Sequencing corroborated the presence of the expected introns in the aberrant unspliced PCR products (Table 5, herein above). These results establish that antifolate-resistance in MTX^{R5}P cells is associated with a splicing defect that impairs the splicing of multiple genes.

DNA Sequencing Reveals Alternatively Spliced FPGS Transcripts in CEM-7OH-MTX Cells

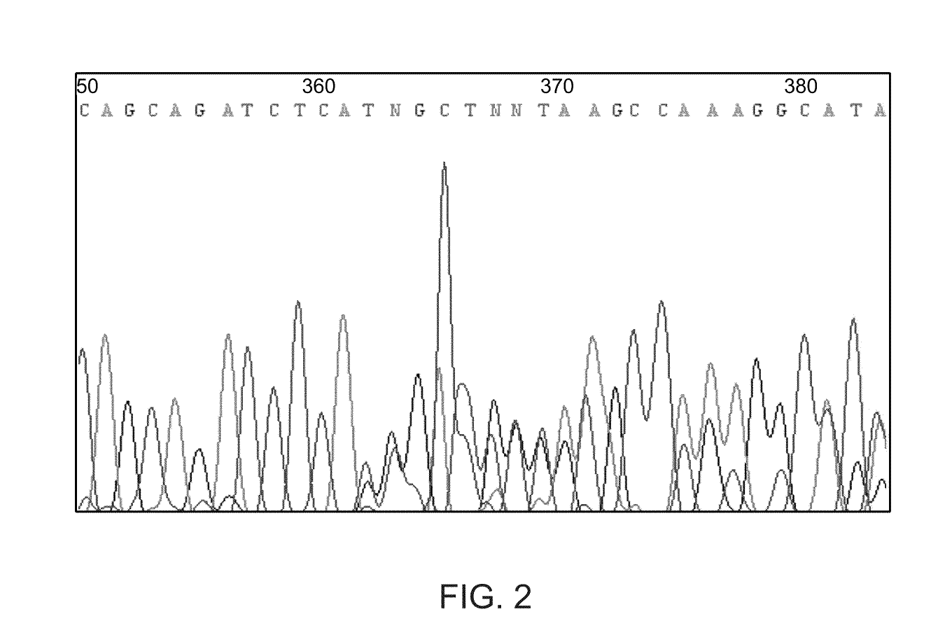

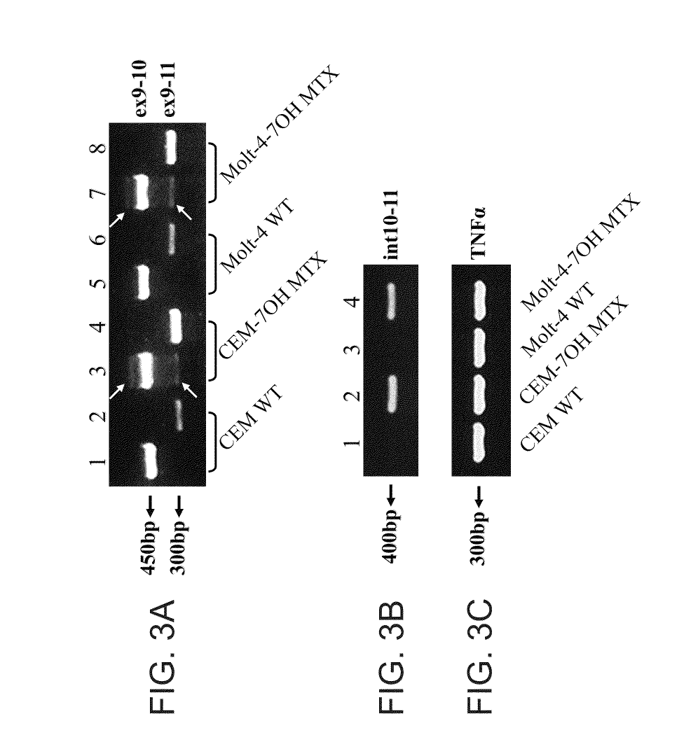

To determine whether impaired splicing of the FPGS gene occurs in other antifolate-resistant human leukemia cell lines, CEM-7OH MTX and Molt-4-7OH MTX cells were used which were recently shown to display 98% loss of FPGS activity [Fotoohi K et al., Blood. 2004; 104:4194-4201]. These cell lines acquired resistance to 7-OH-MTX, the primary metabolite of MTX. Thus, FPGS cDNAs from these cell lines were first screened for point mutations and splicing alterations by PCR (primers are depicted in Table 3, herein above) and then sequenced. DNA sequencing of the PCR product obtained with primers EX8-up (SEQ ID NO: 5) and EX13-dw (SEQ ID NO: 6) (Table 3) revealed the existence of two different transcripts differing in the presence or absence of exon 10 (FIG. 2). Since the nucleotide signal peaks of the aberrant transcript (i.e. that lacks exon 10) were either equivalent or higher than those of the expected transcript, it was hypothesized that this was a major phenotype of alternative splicing. Following this observation, diagnostic primers were specifically designed to PCR amplify FPGS transcripts that contain or lack exon10. Thus, half of the forward primer resides in the 3′-end of exon 9, whereas the other half maps either to the beginning of exon 10 or exon 11 (i.e. primers ex9-10 (SEQ ID NO: 12) and ex9-11 (SEQ ID NO: 13), respectively; see Table 4). PCR performed on both CEM-7OH MTX and Molt-4-7OH MTX cells resulted in high levels of PCR products lacking exon 10 (FIG. 3A, lanes 3 and 4 as well as 7 and 8), whereas parental cells exhibited residual levels of this exon 10-free product (FIG. 3A, lanes 2 and 6). PCR performed on these mutants' cDNA resulted in multiple bands (FIG. 3A, lanes 3 and 7, white arrows), thereby suggesting the presence of additional splicing alterations in the FPGS transcript. The upper most band containing an additional ˜100 bp, may be indicative of the presence of intron 10 which is 95 bp long. To confirm the presence of intron 10 in some of the FPGS transcripts in these splicing defective cell lines, a diagnostic primer was designed, half of which resides in the 3′-end of exon 10 and the other half on the 5′-end of intron 10 (i.e. primer ex10-int10; see Table 4). As evident from FIG. 3B, no visible product is obtained in parental cell lines (FIG. 3B, lanes 1 and 4), whereas antifolate-resistant cells exhibit the predicted product containing intron 10 (FIG. 3B, lanes 2 and 4). DNA sequencing performed on all PCR products confirmed the presence of intron 10. Although FPGS splicing is not completely deficient, the present inventors PCR amplified the TNFα gene in order to explore whether or not other genes remain intact. Using the same primer set as in the MTX^{R5}P cells, a normal PCR product size of TNFα was obtained (FIG. 3C).

Northern Blot Analysis Reveals the Absence of a Principal FPGS Transcript in CEM-7OH MTX Cells

In CEM-7OH MTX and Molt-4-7OH MTX leukemia cells, FPGS activity was decreased by at least 98%, whereas no quantitative alteration was observed at the FPGS mRNA level [Fotoohi K et al., Blood. 2004; 104:4194-4201].

Based on the present findings regarding the defective splicing of the FPGS gene in MTX^{R5}P cells, the present inventors hypothesized that RT-PCR may misrepresent the actual FPGS mRNA levels as a result of the specific position of the primers on the FPGS cDNA. Therefore, the present inventors performed Northern blot analysis to determine quantitatively and qualitatively the actual levels and sizes of the FPGS transcripts in these 7OH MTX-resistant mutants. As evident from FIG. 4A, no discrete FPGS transcript could be detected in CEM-7OH MTX cells (lane 2); instead, a smear spanning several hundreds of base pairs was apparent. Moreover, MTX^{R10} cells, which retained only 1.2% of parental FPGS activity (Table 6) displayed an extended smear as well as a band, though being slightly lower in size than the expected principal FPGS transcript (FIG. 4A lane 3).

The Truncated Form of FPGS Due to Exon 10 Skipping has No Dominant Negative Effect on the Activity of the wt FPGS

The absence of exon 10 from the FPGS transcript in CEM-7OH MTX and Molt-4-7OH MTX cells introduces both a frame shift and a premature stop codon which may result in a truncated FPGS protein containing only 334 amino acids. Since equal levels of FPGS transcripts that contain or lack exon10 were found, the present inventors further determined whether this truncated protein is capable of modulating the activity of the normal FPGS protein. Stable transfectants were generated expressing the FPGS gene either with or without exon 10 in both parental CCRF-CEM and MTX^{R10} cells. Expression levels of these exogenous genes was verified by Northern blot analysis (FIG. 4C), following which FPGS activity assay and antifolate cytotoxicity experiments were undertaken. Ectopic overexpression of the normal FPGS in MTX^{R10} cells resulted in an 800% increase in FPGS activity relative to parental cells and consequently fully restored parental cell sensitivity to ZD1694. In contrast, the truncated form of FPGS lacking exon 10 had no effect on FPGS activity. Moreover, expressing this truncated form in parental cells failed to alter antifolate sensitivity or the catalytic activity of the endogenous FPGS.

ALL Specimens Exhibit FPGS Exon 12 Skipping

To explore the clinical relevance of the current findings of defective FPGS splicing in antifolate-resistant leukemia cell lines, an initial study was undertaken aimed at screening for alterations in FPGS splicing in 13 RNA samples previously obtained from 8 ALL patients, five of which were matched samples at diagnosis and relapse. PCR analysis was designed to confirm the presence of all 15 exons and the absence of all introns (excluding introns 4 and 14 which are too long to be amplified using conventional PCR methodology). As depicted in FIG. 5A, while using a primer set residing within exons 9 and 13, one bone marrow sample from a relapse ALL patient (sample R1) that underwent HD MTX-containing chemotherapy, exhibited a low molecular weight FPGS transcript (FIG. 5A, lane 3), that was absent at the time of diagnosis (sample D1, lane 2). DNA sequencing of this PCR product revealed the absence of FPGS exon 12. A second PCR was performed (FIG. 5B) with the primer ex11-13 (Table 4) which can only anneal to FPGS cDNA lacking exon 12, thereby producing a 250 bp product that is diagnostic of exon 12 skipping. Consistent with the above results, relapse sample R1 exhibited this 250 bp product which was absent at the time of diagnosis (i.e. sample D1), thereby verifying FPGS exon 12 skipping at relapse (FIG. 5B, lanes 2 and 3, respectively). Moreover, two diagnosis specimens (FIG. 5B, D5, D8, lanes 10 and 14 respectively) also exhibited low levels of the 250 bp product. Hence, of the 8 ALL patient specimens analyzed, 3 harbored FPGS exon 12 skipping that results in premature translation termination.

Conclusions

The present examples provide the first evidence that aberrant splicing of FPGS mRNA including intron retention and/or exon skipping, results in premature translation termination, loss of FPGS activity and consequent antifolate-resistance in human leukemia cell lines. The present inventors studied 13 ALL specimens from 8 different patients, 5 of which were matched samples obtained both at the time of diagnosis and at relapse, along with 3 additional diagnosis samples. This preliminary study identified exon 12 skipping in FPGS transcripts both at diagnosis and relapse, thereby suggesting a possible role in the acquisition of ALL resistance to HD MTX (3 gr/m2).

MTX^{R5}P, the first leukemia cell line that was found here to harbor an FPGS splicing defect, retained introns 1, 2, 10, 11 and 12. It should be noted that the presence of the first intron alone in the FPGS transcript is sufficient to introduce a premature stop codon after a 74 amino acids-long open reading frame. The latter would certainly lead either to a truncated FPGS polypeptide which may undergo degradation and/or result in the activation of the nonsense-mediated mRNA decay (NMD) pathway. NMD is an established quality control mechanism which selectively degrades mRNAs harboring premature translation termination codons and thereby prevents their translation. Moreover, even if such a truncated FPGS polypeptide did exist, it would be devoid of FPGS activity as it lacks the C-terminal domain that contains both the binding sites for folate and glutamate. Hence, the present findings provide the first direct evidence associating antifolate-resistance with aberrant FPGS splicing. Moreover, the latter further offers a novel molecular basis for the frequent emergence of tumor cells with antifolate-resistant phenotypes that display apparently normal FPGS mRNA levels commonly evaluated by RT-PCR analysis, while exhibiting loss of FPGS activity in the absence of inactivating point mutations. In this respect, it has been frequently shown that loss of FPGS activity occurs in the absence of a parallel decrease in mRNA levels in multiple CCRF-CEM leukemia cells with acquired resistance to various polyglutamatable antifolates; including MTX, raltirexed, edatrexate or lometrexol (DDATHF). This finding strongly suggested the existence of alterations at the post-transcriptional level of the FPGS gene. In the current study another post-transcriptional alteration was discovered, involving splicing defects in FPGS mRNA leading to loss of FPGS activity. However, whereas quantitative RT-PCR analysis, particularly with non-diagnostic PCR fragments, may frequently fail to identify such splicing defects, Northern blot analysis proved instrumental in identification of changes in the full-length FPGS transcripts suggesting putative aberrant splicing (FIG. 4A); this was corroborated with diagnostic PCR analysis.

Intermittent exposure of tumor cell lines to polyglutamatable-antifolates including MTX, best resembles the clinical setting when a folate antagonist is administered as a pulse (i.e. bolus infusion) and the antifolate monoglutamate is cleared rapidly from the blood. In this respect, a substantial body of literature with multiple human hemato-lymphoid and carcinoma cell lines established that a predominant mechanism of resistance to polyglutamatable antifolates upon a repeated pulse exposure to high dose drug, is decreased FPGS activity and consequently defective antifolate polyglutamylation. In this respect, McGuire et. al. [Oncol Res 1995; 7:535-543] demonstrated that impaired antifolate polyglutamylation can rapidly emerge in CCRF-CEM leukemia cells following intermittent exposure to MTX via positive selection of pre-existing clones with decreased FPGS activity. The major mechanism of antifolate-resistance in these clonal isolates was diminished FPGS activity and subsequently impaired MTX polyglutamylation. Remarkably, even after a single pulse exposure to MTX, decreased FPGS activity, defective polyglutamylation and consequent antifolate resistance were apparent; consistently, FPGS activity gradually decreased with the number of intermittent MTX treatment cycles. These findings of positive selection of pre-existing clonal variants with decreased FPGS activity in CCRF-CEM cells are in accord with the current findings of aberrant FPGS splicing in the very same leukemia CCRF-CEM cells. It was consistently found that the population of untreated parental CCRF-CEM cells had some small, yet detectable alteration in the splicing of FPGS mRNA including exon 10 skipping (FIG. 3A) as well as intron 10 retention; these alterations became dramatic in the antifolate-resistant sublines. Based on these cumulative results, a plausible scenario that can be envisioned is that pre-existing clonal variants displaying some basal, low level of aberrant splicing of FPGS mRNA may exist in the leukemia cell population prior to treatment with MTX. Under antifolate-free growth conditions, these pre-existing variants are relatively infrequent in the general population as they harbor a growth disadvantage due to their inability to efficiently form and accumulate folate polyglutamates, thereby resulting in a contracted intracellular folate pool. However, upon repeated cycles of pulse drug exposure to relatively high concentrations (i.e. clinically relevant) of polyglutamatable antifolates, these rare clones may be rapidly selected out from the general population. This is particularly true as these clones bear a growth advantage under drug selective conditions due to their inability to form antifolate polyglutamates, hence resulting in rapid efflux of antifolate monoglutamates.

MTX undergoes oxidation to its principal metabolite 7-hydroxymethotrexate (7-OHMTX) in the liver by aldehyde oxidase. The plasma concentration of 7-OHMTX can exceed that of MTX, as in osteosarcoma and ALL patients treated with HD MTX, thereby becoming the predominant metabolite 10-12 hr after bolus MTX infusion. Recently the present inventors have shown that MTX and its metabolite 7-OHMTX provoke disparate mechanisms of antifolate resistance in leukemia cells [Fotoohi K et al., Blood. 2004; 104:4194-4201]; hence, whereas continuous exposure to MTX leads to down-regulation of RFC expression and markedly impaired drug uptake, exposure to 7-OHMTX brought about a major decrease (˜98%) in FPGS activity with no apparent quantitative alteration at the mRNA level [Fotoohi K et al., Blood. 2004; 104:4194-4201]. These findings suggest the involvement of FPGS and not RFC in cases of relapse following treatment with MTX. In contradistinction to Fotoohi et al, the present inventors here show that the level of the principal transcript of FPGS is down-regulated as no specific band could be observed in these cell lines upon Northern blot analysis (FIG. 4A). Instead of a discrete principal transcript band, these antifolate-resistant cells exhibited a smear extending for several hundreds of base pairs insinuating the existence of multiple FPGS transcripts differing in length, thereby reflecting the retention of introns and/or the skipping of exons.

In order to explore the possibility of the occurrence of splicing defects in the FPGS transcript in the clinical setting, the present inventors obtained 13 RNA samples from 8 different ALL patients, 5 of which were matched specimens obtained both at diagnosis and relapse. PCR amplification of a region of the FPGS transcript encompassing exons 9-13 established exon12 skipping in one relapse specimen (FIG. 5A, lane 3); the ratio between the relatively low level of the 300 bp PCR product reflecting exon 12 skipping and the high level of the normal (i.e. properly spliced) 450 bp product was found to be in a good concordance with the ratio of leukemic cells vs. normal leukocytes in the bone marrow from which the RNA sample was derived (i.e. patchy infiltration of leukemia cells among normal hematopoietic cells in a normocellular bone marrow). Taking into consideration the fact that complete remission (CR) is determined in the presence of less than 5% blasts in a normocellular bone marrow, patients with a smaller extent of disease might be erroneously determined as achieving a promising CR. Interestingly, 93% of all adult ALL patients achieve CR according to these criteria, whereas less than 40% eventually remain free of disease. Hence, a relatively low level of the aberrant PCR product reflects the poor ratio of blast cells to normal cells, rather than reflecting mere low levels of the aberrantly spliced FPGS transcript relative to the normal transcript. To corroborate these findings, a second diagnostic PCR was performed that exclusively amplified the aberrant FPGS transcript that lacks exon 12 (FIG. 5B); this sensitive PCR approach allowed the present inventors to positively identify two additional ALL diagnosis specimens that also contained aberrant FPGS transcripts lacking exon 12. A second specimen obtained at diagnosis (D5) (FIG. 5B, lane 10) exhibited very low levels of the aberrant FPGS transcript which was absent at the time of relapse (FIG. 5B, lane 11), whereas specimen D8 (FIG. 5B, lane 14) had an elevated level of exon 12 skipping, suggesting a pre-existing alteration in FPGS which could contribute to drug-resistance following the HD-MTX-containing combination chemotherapy via positive clonal selection and expansion. Indeed, the latter patient succumbed shortly after the HD-MTX-containing chemotherapy due to a rapidly progressing disease. In the antifolate-resistant leukemia cell lines the present inventors identified exclusion of FPGS exon 10 as well as inclusion of intron 10, each of which introduced premature stop codons that could activate the NMD pathway, and in addition, disrupt the open reading frame such that the glutamic acid binding site composed of D335, H338 and R377 is completely absent. In the clinical ALL specimens, exon 12, however, encodes for D335 and H338; hence, exon 12 skipping eliminates glutamic acid binding as well as introduces a premature stop codon. The existence of an FPGS transcript lacking exon 12 in 3 out of 8 ALL patients raises the possibility of using it as a potential molecular tool for the prediction of the response of leukemic cells to MTX.

### Example 2

### Folylpoly-γ-Glutamate Synthetase Splicing Defects are Highly Correlated with Poor Prognosis in Adult Acute Lymphoblastic Leukemia

Materials and Methods

Study design: The analysis was restricted to specimens that met the following criteria: 1) Patients whose diagnosis BM or PB specimens underwent RNA analysis and for whom sufficient amount of stored RNA was available. 2) Patients treated between the years 2003 and 2007 that could ensure a good quality of stored RNA necessary to achieve a sufficient follow up, allowing for a reliable assessment of survival parameters. PB was also obtained from 8 healthy volunteers, of which 4 were adults and 4 were young adults between the ages of 30 and 50 (half females and half males). Patients' clinical data was documented by primarily focusing on risk factors at diagnosis; i.e. age >35 years and high WBC count of ≧35,000 and ≧100,000 for B-ALL and T-ALL, respectively; poor cytogenetics; t(9; 22) and t(4; 11), response to chemotherapy, remission duration, and long-term OS. Demographic characteristics were further verfied—(gender and age), ALL subtype, divided into T ALL, Ph⁺ B ALL, and Ph⁻ B ALL, and therapy applied (i.e., protocol combination regimen and stem cell transplantation. 24 patients in all were analyzed (8 of which were used in Example 1). RNA samples were obtained at the time of diagnosis as part of the routine clinical management.

Assay of FPGS splicing defects: RNA was derived from leukocytes isolated either from PB or BM, using a standard Ficoll-Hypaque density centrifugation. Total RNA was purified using the Tri-Reagent kit according to the instructions of the manufacturer (Sigma) and reverse transcribed. Using RT-PCR, specimens were screened for FPGS exon skipping using novel diagnostic primers specific for the skipping of exons 3, 7, 8 or 12 (i.e. which can anneal solely to cDNA lacking exon 3, 7, 8 or 12); EX2-4 5′-GAGTGGGCTGCAGGGCTC-3′ (SEQ ID NO: 41) with Ex6-dw 5′-AGGCCATGAGTGTCAGGAAGC-3′(SEQ ID NO: 42); ex6-8 sense: 5′-CCAAGAGAAGGAAGCCTGTG-3′ (SEQ ID NO: 43) used with 4R: 5′-AGCATCGGACACAGGTATAGA-3′ (SEQ ID NO: 44), ex7-9 sense: 5′-GCACCAACATCATCAGCAAGG-3′ (SEQ ID NO: 45) used with 6R 5′-CGGTCCCCGGTAGCATTGA-3′ (SEQ ID NO: 46) and ex11-13 [SEQ ID NO: 16] used with EX15-dw [SEQ ID NO: 8].

Control reactions were performed using the following primers: EX1-up [SEQ ID NO: 1] with EX3-dw [SEQ ID NO: 2] and 7F (5′-AGGCCTGCGTGCGCTGGTT-3′) with EX15-dw [SEQ ID NO: 8] for FPGS exons 1-3 and 12-15, respectively, as well as GAP-up with GAP-dw [SEQ ID NOs; 31 and 32] for GAPDH exons 6-8. As mentioned above, this analysis was also performed on PB samples obtained from healthy volunteers.

Statistic analysis; Progression-free survival (PFS) was defined as time from complete remission (CR) to recurrence or death. Overall survival (OS) was defined as the time from diagnosis to death from any cause, while death of disease (DOD) required death during disease progression. Surviving patients were censored at the latest follow-up for all parameters. Survival analysis was performed using the Kaplan-Meier product limit estimation and differences between subgroups were analyzed using Log Rank test. Associations between categorical variables were tested using the Fisher's exact test. Two-tailed p values ≦0.05 were considered to be statistically significant.

Results

Clinical data of patients: The characteristics of the 24 ALL patients studied are depicted in Table 7, herein below.

TABLE 7
Patients' characteristics at diagnosis

| | Whole series | |
|---|---|---|
| Parameter | (n = 24) | |
| Sex, male | 14 | |
| Age, y-median | 40.5 | (18-72) |
| (range) | | |
| WBC, cell/μl-median | 40,000 | (990-423,000) |
| (range) | | |

| | |
|---|---|
| ALL type | |
| T | 5 |
| B, | 8 |
| Ph+ | |
| B, | 11 |
| Ph* | |
| Treatment protocol | |
| ECOG 2993/UKALL12 | 23 |
| Other protocol** | 1 |
| Allogeneic SCT | 11 |

*1 harbored t(4; 11)

Median age at diagnosis was 40.5 years (ranging between 18 and 72 years). Fifteen patients (62%) were older than 35 years, 13 had a high WBC count and 9 had poor risk karyotype (eight were Ph⁺ ALL and one had t(4; 11)). Patients were treated according to the UKALL12/ECOG 2993 protocol [Goldstone et al., 2008, Blood 111, 1827-1833], which included two induction phases (completed on day 56), followed by intensification phase consisting of high dose MTX. Younger Ph(+) ALL patients and those with Ph(−) ALL who had a matched sibling donor were referred to an allogeneic stem cell transplantation (SCT) in first CR. Younger patients with Ph(−) ALL who lacked a matched sibling donor were randomized for chemotherapy versus autologous SCT. Patients older than 60 years continued chemotherapy irrespective of the Ph status. Three of the 24 patients died before completing the induction phase, hence they were not available for assessment of disease response. Seventeen patients achieved CR and continued the protocol, receiving high dose MTX followed by allogeneic SCT (n=9), chemotherapy (n=9), or autologous SCT (n=1). Four patients never responded and received several chemotherapeutic regimens, including high dose MTX followed by allogeneic SCT (n=2). Within a median follow-up of 36 months for survivors, 7 patients are alive and free of disease, whereas 17 patients had died. Fourteen patients died of disease, of which 3 died shortly after diagnosis, 4 due to disease progression and 7 due to a non-responding relapse.

Identification of FPGS Splicing Defects in Samples from Diagnosis ALL Patients and Their Correlation with Patients' Outcome

After verifying equal expression levels of FPGS and GAPDH with previously described primers (FIG. 6B), analysis of FPGS splicing defects was performed by RT-PCR on diagnosis ALL patients' specimens using primers specific for the skipping of either exons 7, 8 or 12. Of the 24 evaluable patients, 11 (46%) exhibited FPGS exon 12 skipping, 7 (30%) displayed FPGS exon 7 skipping (one patient was excluded being non-informative for exon 7 due to limited amounts of RNA) and 6 (25%) harbored both splicing defects (FIG. 6A), whereas none of the patients exhibited skipping of exon 8 (data not shown); in all, 12 patients harbored a splicing defect. In contrast, no instances of FPGS exon 7, 8 or 12 skipping were observed in samples obtained from healthy individuals (data not shown).

Exon 3 skipping was also demonstrated in ALL patients. It should be emphasized that exon 3 contains the ATP binding fold and hence omission of this exon results in complete loss of FPGS activity (Zhao, R. et. al. 2000. JBC).

The statistic significance of the possible association between FPGS exon skipping (i.e. defective FPGS splicing) and disease outcome in each patient was explored (Tables 8 and 9, herein below).

TABLE 8

| | | | | Patients | | |
|---|---|---|---|---|---|---|
| | | | | negative | | |
| | | Patients | | for | | |
| | | positive for | | splicing | | |
| | | splicing | | defects | | |
| Parameter | | defects* (n = 12) | | (n = 11)** | | P |
| Sex, male | | 5 | | 8 | | N.S. |
| Age, y | Median (range) | 44.5 | (19-68) | 28 | (18-72) | |
| | Age >35 years, n | 10 | (83) | 4 | (36) | 0.036 |
| | (%) | | | | | |
| WBC, | Median (range) | 96,000 | (40,000-423,000) | 14,830 | (990-166,000) | |
| cell/μl | | | | | | |

| | | | | | |
|---|---|---|---|---|---|
| High >35,000 for B-ALL or | 9 | (75) | 4 | (36) | N.S. |
| >100,000 for T-ALL, n (%) | | | | | |

| | | | | |
|---|---|---|---|---|
| ALL type | T | 2 | 3 | |
| | B, Ph+ | 4 | 4 | |
| | B, Ph−*** | 6*** | 4 | |
| | Poor | 5 | 4 | N.S. |
| | karyotype**** | | | |
| Treatment | ECOG | 11 | 11 | |
| protocol | 2993/UKALL12 | | | |
| Allogeneic | | 7 | 4 | |
| SCT | | | | |

P values were calculated using the Fisher's exact test
*Skipping of exons 7 and/or 12 of the FPGS gene
**1 patient was not evaluable for the analysis of exon 7 skipping
***1 harbored t(4; 11)
****Ph+ or t(4; 11)

Three out of 24 patients died before completing the induction phase and therefore, were not available for assessment of disease response but were informative for OS. Patients harboring skipping of FPGS exons 7 and/or 12 were older (>35 years, 83.3%, p=0.036), however, they neither had a higher incidence of poor risk cytogenetic abnormalities (p=1) nor presented with higher WBC counts (p=0.1; Table 8). The outcome of the patients presenting with the skipping of FPGS exon 7 and/or 12 was significantly worse than that observed for patients lacking these splicing defects, as detailed below (FIG. 7, Table 9): Patients displaying skipping of both FPGS exons 7 and 12 had an extremely poor response rate; 4/6 of these “double positive” patients had a chemo-refractory disease and never achieved a CR, whereas ½ of these “double positive” patients that did achieve a CR had a disease progression a month after receiving high dose MTX-containing chemotherapy.

TABLE 9

| | | All | | | | | | | | | | | | |
|---|---|---|---|---|---|---|---|---|---|---|---|---|---|---|
| Prog- | | Evalu- | | | | | | | PFS**, *** | | | DOD*** | | |
| nostic | | able, | | CR* | | | Relapse** | | Medi- | | | median, | | |
| Factor | | N (%) | | n (%) | | P | n (%) | | an, m | % | P | m | % | P |
| No. | | 24 | (100) | 17 | (81) | — | 9 | (53.5) | 11 | 29 | — | 14.5 | 54 | — |
| Age, y | <35 | 9 | (37.5) | 6 | (75) | N.S. | 3 | (50) | 17 | 50 | N.S. | H.B.R. | 53 | N.S. |
| | >35 | 15 | (62.5) | 11 | (78.5) | | 6 | (54.5) | 18 | 24 | | 11 | 18 | |
| WBC, | low | 11 | (46) | 9 | (100) | N.S. | 3 | (33) | 25 | 48 | 0.012 | 29 | 45 | N.S. |
| cell/μl | high\hat{ } | 13 | (54) | 8 | (67) | | 6 | (75) | 6 | 19 | | 10.5 | 19 | |
| Karyo | Stan- | 15 | (62.5) | 11 | (85) | N.S. | 5 | (45.5) | 18 | 47 | N.S. | 19 | 39 | N.S. |
| type | dard | | | | | | | | | | | | | |
| | Poor | 9 | (37.5) | 6 | (75) | | 4 | (75) | 17 | 0 | | 29 | 0 | |
| | \hat{ }\hat{ } | | | | | | | | | | | | | |
| Exon | No | 11 | (48) | 10 | (100) | N.S. | 3 | (30) | H.B.R. | 65 | 0.015 | H.B.R. | 78 | 0.001 |
| Skip- | \hat{ }\hat{ }\hat{ } | | | | | | | | | | | | | |
| ping | 7 | 12 | (52) | 6 | (60) | | 5 | (83) | 4 | 0 | | 10.5 | 0 | |
| | and/ | | | | | | | | | | | | | |
| | or 12 | | | | | | | | | | | | | |
| Allo | yes | 11 | (46) | 9 | (82) | N.D. | 4 | (44) | 18 | 33 | N.S. | H.B.R. | 58 | 0.03 |
| SCT | no | 13 | (54) | 8 | (80) | | 5 | (62.5) | 17 | 43 | | 10.5 | 20.5 | |

P values were calculated using either Fisher's exact test or the Log Rank test.
**N = 16, (6 for the positive group) only patients who achieved remission are assessed for relapse and PFS.
***At last follow-up, median time for survivors 36 months.
\hat{ }>35,000 for B-ALL or >100,000 for T-ALL.
\hat{ }\hat{ }8 patients with Ph+ and one with t(4; 11).
\hat{ }\hat{ }\hat{ }1 patient was not evaluable for the analysis of exon 7 skipping.

In contrast, 10/10 patients lacking these FPGS splicing defects, achieved a CR (Table 9). Patients presenting with these FPGS splicing abnormalities also experienced a significantly higher progression rate; in addition to the 4 patients who never achieved remission and 2 patients that died before completing the induction phase, 5/6 remaining patients rapidly experienced disease relapse (median time to relapse was 4 months). In contrast to the dismal outcome observed in patients harboring FPGS splicing defects, patients who were devoid of these FPGS alterations experienced significantly longer remissions (median time to relapse has not been reached, p=0.015) (FIG. 7A); 3 of these 10 patients experienced relapses 6, 11 and 18 months after diagnosis, one of which died of disease progression, while two were salvaged with allograft transplantation and achieved durable remissions. The risk of disease-related death was found to be significantly higher in patients diagnosed with the skipping of FPGS exon 7 and/or 12 (0% survival at last follow-up, with a median survival time of 10.5 months) than in patients with normal FPGS splicing (78% survival at last follow-up, and median survival time has not been reached, p=0.001) (FIG. 7B). Consistently, the OS rates of patients harboring FPGS splicing defects were significantly lower (0% survival at last follow-up, with a median survival time of 7.5 months) than those with normal splicing (62% survival at last follow-up and median survival time has not been reached thus far, p=0.0008) (FIG. 7C).

When evaluating the correlation between known prognostic factors and disease progression, almost no significant correlation was observed; poor cytogenetic karyotype was insignificantly associated with achieved remission, occurrence of relapse, disease-related death and OS (p=0.61, 0.6, 0.77 and 0.9 respectively). The same applies to the factor of >35 years of age (p=0.61, 0.5, 0.29 and 0.07 respectively). High WBC counts showed significant association solely with short progression-free survival (PFS) (19% of disease free rates at the end of follow-up vs. 48% for low WBC count, p=0.012) (FIG. 7A). Conversely, therapy by allogeneic SCT was highly associated with risk of disease-related death (58% of survival rates at the end of follow-up vs. 20.5% for patients that did not undergo SCT, p=0.03) (Table 9). In addition, the above analyses were repeated while restricting the study to the 15 patients with standard risk karyotype. Age was the only standard prognostic factor to exhibit significant association with OS (p=0.032). FPGS splicing defects, however, were highly associated with patient outcome; 86% death of disease vs. 20% (p=0.0017) and 0% OS vs. 57% (p=0.0018).

Conclusion

FPGS exon skipping as a single, novel prognostic marker displayed excellent correlation with patient outcome; exon skipping was highly correlated with the occurrence of relapse (100%, p=0.015), disease-related death (100%, p=0.001) and OS (0%, p=0.0008), with no patients surviving at the end of follow-up. The median times to relapse and to die from disease are very short for patients harboring exon skipping (i.e., 4 and 7.5 months, respectively) while the median times for the negative group have not been reached. A patient positive for both FPGS splicing defects had a high risk of refractory disease (75%), although, a single exon skipping abnormality (i.e. either 7 or 12) was sufficient to predict certain death (p=0.0018 or 0.0012, respectively) after a median survival time of 10.5 months. The present inventors evaluated the correlation between FPGS exon skipping and other prognostic factors. It was found that patients harboring skipping of FPGS exons 7 and/or 12 were older (>35 years, 83.3%, p=0.036), however, they did not have a higher incidence of poor risk cytogenetic abnormalities (p=1) nor presented with higher WBC counts (p=0.1). It should be emphasized that the statistical tests used in the current study to analyze the significance of the present results were carefully selected and are best suited to evaluate small sample size (i.e. relatively small number of ALL patients). Indeed, the various P-values obtained in the current study, when analyzing the statistical significance of the present data with 24 adult ALL patients, were highly significant with the novel parameter of impaired FPGS splicing (p values ≦0.015). Another point attesting to the genuine significance of our FPGS splicing data in ALL patients at diagnosis is the fact that a small sample size typically increases the P-value (i.e. decreases the significance) which was completely not the case with the statistically significant impaired FPGS splicing results. In contrast, the other prognostic factors studied herein did not reach statistical significance. Therefore, the fact that the novel parameter of impaired FPGS splicing achieved highly significant p-values strongly attests that the results obtained with this prognostic factor are authentic and definitely not affected by the relatively small number of ALL patients studied here. Taken together, the novel genetic marker of FPGS splicing defects devised here for the assessment of adult ALL prognosis proved superior over established prognostic factors, including high WBC count, age >35 years and poor cytogenetic abnormalities. Moreover, this molecular prognosis marker assay is rapid (can be completed in a few hours), facile, reliable and inexpensive. It is noteworthy, that no false-positives (i.e., patients harboring exon skipping with good outcome) were found and a low percentage of false negatives (i.e., patients having no exon skipping with disease progression, 30%). The existence of false negatives could be explained, in part, by low blast content in PB (i.e., two patients had only 2% and 3% blasts). Upon future clinical implementation of an FPGS splicing defect-based prognostic assay, it would be desirable to use BM specimens rather than PB, thereby, alleviating the variability in the WBC content. Moreover, it is very likely that the skipping of other exons which were not explored here are present in these negative patients; hence further exploration of the spectrum of FGPS splicing defects is warranted.

The detection of FPGS splicing defects may prove vital for patients with standard risk karyotype for whom the role of allogeneic SCT in first CR is still under debate. The presence of FPGS splicing defects segregates “standard risk karyotype patients” into favorable and unfavorable subgroups (after 2 years, the percentages of death from disease were 20 vs. 86, respectively, p=0.0017), suggesting that assessment of FPGS splicing defects could potentially be a useful tool in tailoring patient treatment.

Although FPGS splicing defects in adult ALL specimens at diagnosis may not have a direct role on drug resistance during the induction phase, it may affect PFS and OS, since MTX is the anchor drug in the intensification phase (after archiving remission). Moreover, the present inventors propose that the mechanism responsible for impaired FPGS splicing is likely to interfere with the splicing of other genes; alternative splicing of an upstream apoptosis-related gene, such as FAS and Bcl-x, may confer multidrug resistance which can be accounted for by the high relapse rates and high percentage of chemo-refractory disease observed in the present study. In addition, a high correlation between FPGS exon skipping and age (>35 years, p=0.036) was observed. The higher incidence of FPGS exon 7 and/or 12 splicing defects in older patients is likely to reflect an increased frequency of splicing defects of various genes with age that is associated with pre-existing genomic instability, the latter of which is the hallmark of cancers. Importantly, no FPGS splicing defects were detected in specimens obtained from both young and elderly healthy individuals. However, although exon skipping is correlated with older age and patients exhibiting this abnormality tend to have high WBC counts, exon skipping is not a surrogate marker of either. This is due to the discrepancy between the statistical significance of the correlation of each factor and the chance of death. While exon skipping was highly correlated with short PFS and OS as well as certain death of disease, high WBC was correlated solely with short PFS, and age >35 years was not significantly correlated with either.

In summary, aberrant splicing in adult ALL may result in increased chemo-resistance and, consequently, rapid disease progression and poor survival prognosis.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of predicting responsiveness of a subject to a folylpolyglutamate synthetase (FPGS) dependent anti-folate, the method comprising analyzing for a presence or absence of a splice variant of FPGS or a polypeptide encoded thereby, in a sample of the subject, wherein said presence of said splice variant or said polypeptide encoded thereby is indicative of a negative response to a FPGS-dependent anti-folate.

2. The method of claim 1, wherein said sample comprises bone marrow or peripheral blood.

3. The method of claim 1, wherein said sample comprises an RNA sample or a protein sample.

4. (canceled)

5. (canceled)

6. The method of claim 1, wherein said analyzing is effected by calculating a size of an RNA encoding FPGS.

7. The method of claim 1, wherein said analyzing is effected by determining a sequence of at least a portion of an RNA encoding FPGS.

8. The method of claim 1, wherein said analyzing is effected using an antibody which binds to said polypeptide encoded by said splice variant of said FPGS and does not bind to a wild-type FPGS.

9. The method of claim 7, wherein said sequence of at least said portion of said RNA encoding FPGS comprises at least a part of an intron selected from the group consisting of intron 1, 2, 10, 11 and 12.

10. The method of claim 7, wherein said sequence of at least said portion of said RNA encoding FPGS is devoid of at least a part of an exon selected from the group consisting of exon 3, exon 7, exon 10 and exon 12.

11. The method of claim 7, wherein said sequence of at least said portion of said RNA encoding FPGS is selected from the group consisting of SEQ ID NOs: 47-51.

12. The method of claim 7, wherein said sequence of at least said portion of said RNA hybridizes with an RNA molecule which comprises a nucleic acid sequence as set forth in SEQ ID NOs: 16, 41 and 43.

13. The method of claim 7, wherein said sequence of at least said portion of said RNA comprises a mutation in at least one of the positions selected from the group consisting of:
(i) a region bridging exon 2 and exon 3 of said FPGS RNA;
(ii) a region bridging exon 3 and exon 4 of said FPGS RNA;
(iii) a region bridging exon 6 and exon 7 of said FPGS RNA;
(iv) a region bridging exon 7 and exon 8 of said FPGS RNA;
(v) a region bridging exon 9 and exon 10 of said FPGS RNA;
(vi) a region bridging exon 10 and exon 11 of said FPGS RNA;
(vii) a region bridging exon 11 and exon 12 of said FPGS RNA; and
(viii) a region bridging exon 12 and exon 13 of said FPGS RNA.

14. The method of claim 1, wherein the subject has been diagnosed with a disease selected from the group consisting of cancer, an inflammatory disease, and an autoimmune disease.

15. (canceled)

16. A method of selecting an anti-folate for the treatment of a disease in a subject in need thereof, the method comprising analyzing for a presence or absence of a splice variant of FPGS or a polypeptide encoded thereby, in a sample of the subject, wherein said presence of said splice variant or polypeptide encoded thereby is indicative of treatment with a FPGS-independent anti-folate and said absence of said splice variant or said polypeptide encoded thereby is indicative of treatment with a FPGS-dependent anti-folate.

17. (canceled)

18. (canceled)

19. The method of claim 16, wherein said analyzing is effected by calculating a size of an RNA encoding FPGS.

20. The method of claim 16, wherein said analyzing is effected by determining a sequence of at least a portion of an RNA encoding FPGS.

21. The method of claim 16, wherein said analyzing is effected using an antibody which binds to said polypeptide encoded by said splice variant of said FPGS and does not bind to a wild-type FPGS.

22. The method of claim 20, wherein said sequence of at least said portion of said RNA encoding FPGS comprises at least a part of an intron selected from the group consisting of intron 1, 2, 10, 11 and 12.

23. The method of claim 20, wherein said sequence of at least said portion of said RNA encoding FPGS is devoid of at least a part of an exon selected from the group consisting of exon 3, exon 7, exon 10 and exon 12.

24. The method of claim 20, wherein said sequence of at least said portion of said RNA encoding FPGS is selected from the group consisting of SEQ ID NOs: 47-51.

25. The method of claim 20, wherein said sequence of at least said portion of said RNA hybridizes under physiological conditions with an RNA molecule which comprises a nucleic acid sequence as set forth in SEQ ID NOs: 16, 41 and 43.

26. The method of claim 20, wherein said sequence of at least said portion of said RNA comprises a mutation in at least one of the positions selected from the group consisting of:
(i) a region bridging exon 2 and exon 3 of said FPGS RNA;
(ii) a region bridging exon 3 and exon 4 of said FPGS RNA;
(iii) a region bridging exon 6 and exon 7 of said FPGS RNA;
(iv) a region bridging exon 7 and exon 8 of said FPGS RNA;
(v) a region bridging exon 9 and exon 10 of said FPGS RNA;
(vi) a region bridging exon 10 and exon 11 of said FPGS RNA;
(vii) a region bridging exon 11 and exon 12 of said FPGS RNA; and
(viii) a region bridging exon 12 and exon 13 of said FPGS RNA.

27. The method of claim 16, wherein the disease is selected from the group consisting of cancer, an inflammatory disease, and an autoimmune disease.

28. (canceled)

29. An antibody which binds to an expression product of a splice variant of FPGS and does not bind to wild-type FPGS.

30. A kit for assessing a responsiveness of a patient to antifolate therapy, the kit comprising at least one polynucleotide agent which detects a splice variant of FPGS RNA.

31. The kit of claim 30, wherein said at least one polynucleotide agent is encoded by a nucleic acid sequence selected from the group consisting of SEQ ID NOs: 16, 41 and 43.

32. (canceled)

33. A kit for assessing a responsiveness of a patient to antifolate therapy, the kit comprising the antibody of claim 29.

34-36. (canceled)
